(19) United States
(12) Patent Application Publication
(10) Pub. No.: US 2026/0290581 A1
(43) Pub. Date: Sep. 24, 2026

(54) TRACKING PATIENT CONDITION SYMPTOMS WITH TEMPERATURE AND IMPEDANCE DATA COLLECTED WITH IMPLANTED SENSOR

(71) Applicant: Medtronic, Inc., Minneapolis, MN (US)

(72) Inventors: Matthew J. Hoffman, St. Paul, MN (US); Val D. Eisele, III, Little Canada, MN (US); Shantanu Sarkar, Roseville, MN (US); Ryan D. Wyszynski, Oak Grove, MN (US)

(21) Appl. No.: 18/996,066

(22) PCT Filed: Jul. 18, 2023
(86) PCT No.: PCT/IB2023/057317

§ 371 Date: Jan. 17, 2025

Related U.S. Application Data
(60) Provisional application No. 63/369,625, filed on Jul. 27, 2022.

(51) Int. Cl. G16H 50/20; G16H 10/40; G16H 50/30

(52) U.S. Cl. CPC G16H 50/20; G16H 10/40; G16H 50/30

(57) ABSTRACT

Techniques for classifying a health condition of a patient are described. An example technique may include utilizing a probability model that uses various diagnostic states of physiological parameters, which may include fluid retention and temperature, to determine a classification of a health condition of the patient. The probability model may determine a probability score indicating a likelihood of the classification of the health condition being correct. The probability model may output the classification of the health condition and the probability score.

This application claims the benefit of U.S. Provisional Patent Application Ser. No. 63/369,625, filed 27 Jul. 2022, the entire content of which is incorporated herein by reference.

### TECHNICAL FIELD

This disclosure relates to medical devices and, more particularly, medical devices for detecting or monitoring patient conditions.

### BACKGROUND

A variety of medical devices have been used or proposed for use to deliver a therapy to and/or monitor a physiological condition of patients. As examples, such medical devices may deliver therapy and/or monitor conditions associated with the heart, muscle, nerve, brain, stomach or other organs or tissue. Medical devices that deliver therapy include medical devices that deliver one or both of electrical stimulation or a therapeutic agent to the patient. Some medical devices have been used or proposed for use to monitor or detect chronic and acute illnesses such as chronic obstructive pulmonary disease (COPD), sepsis, infection, and heart failure (HF).

### SUMMARY

This disclosure describes techniques for providing an early warning for various health or heart conditions (e.g., COPD, sepsis, infection, HF decompensation, worsening HF, or other cardiovascular-related conditions, such as edema). The disclosed techniques use prediction and probability modeling to classify (e.g., determine a classification of) a heart condition of a patient. In this manner, the disclosed techniques may allow detection or prediction of such events, e.g., even if there are no physical manifestations apparent. The probability or likelihood classification may include a probability score indicating a likelihood of the classification of the health condition being correct.

Furthermore, the disclosed techniques include calibration of physiological parameters, such as impedance and temperature, for classifying the health condition. For example, calibration may involve measuring the physiological parameters when the physiological parameters values are relatively stable (e.g., when a patient is resting but not when the patient is exercising), correcting values of the physiological parameters based on information transmitted from another device, such as an external device, applying a feature extraction model, and so on. Calibration of the physiological parameters in this way may improve the accuracy of classifications. Thus, calibration may increase the reliability or trustworthiness of detections and predictions of health conditions.

Detecting trends in physiological parameters, such as temperature and impedance, and making such data available to patients and clinicians may provide one or more advantages. For instance, physiological parameter trends may provide patients with a key health metrics notifying the patient of an onset of an adverse health condition (e.g., an infection, HF, a virus, etc.). In other words, the physiological parameter trends may represent key precursors that help identify patients in need of treatment. Additionally, collection of physiological parameter values may enable remote monitoring (e.g., monitoring outside of a hospital setting), which may be important for managing some patients (e.g., patients with cardiac comorbidities). For instance, the techniques may enable the implementation of alerts that facilitate early treatment of health conditions, such as HF.

Thus, the techniques of this disclosure may enable earlier detection and treatment of health conditions, which may be imperative to reduce the risk of disease progression. For instance, tracking patient temperature through a subcutaneous implant may aid clinicians in the early detection and management of maladies like influenza and other diseases that have been shown to increase the likelihood of AF, heart attacks and stroke in patients with pre-existing heart disease.

In some examples, a medical system includes: an implantable medical device; an external device; a data server; and processing circuitry configured to: determine a respective one or more values for each of a plurality of physiological parameters, the plurality of physiological parameters comprising: fluid retention, wherein the respective one or more values for fluid retention correspond to a fluid retention state from a first set of states comprising a plurality of fluid retention states; and temperature, wherein the respective one or more values for temperature correspond to a temperature state from a second set comprising a plurality of temperature states; determine, using a probability model, one or more diagnostic states based on the respective values; determine a classification of a health condition of a patient based on the application of the diagnostic states to the probability model; determine, from the probability model, a probability score indicating a likelihood of the classification of the health condition being correct; and generate an output based on the health condition and the probability score.

In some examples, a method includes: determining, by processing circuitry of a medical device system, a respective one or more values for each of a plurality of physiological parameters, the plurality of physiological parameters comprising: fluid retention, wherein the respective one or more values for fluid retention correspond to a fluid retention state from a first set of states comprising a plurality of fluid retention states; and temperature, wherein the respective one or more values for temperature correspond to a temperature state from a second set comprising a plurality of temperature states; determining, by the processing circuitry, one or more diagnostic states based on the respective values; determining, by the processing circuitry, a classification of a health condition of a patient based on the application of the diagnostic states to the probability model; determining, by the processing circuitry and from the probability model, a probability score indicating a likelihood of the classification of the health condition being correct; and generating, by the processing circuitry, an output based on the health condition and the probability score.

In some examples, a computing device includes processing circuitry configured to: determine a respective one or more values for each of a plurality of physiological parameters, the plurality of physiological parameters comprising: fluid retention, wherein the respective one or more values for fluid retention correspond to a fluid retention state from a first set of states comprising a plurality of fluid retention states; and temperature, wherein the respective one or more values for temperature correspond to a temperature state from a second set comprising a plurality of temperature states; determine, using a probability model, one or more diagnostic states based on the respective values; determine a classification of a health condition of a patient based on the application of the diagnostic states to the probability model; determine, from the probability model, a probability score indicating a likelihood of the classification of the health condition being correct; and generate an output based on the health condition and the probability score.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a conceptual diagram illustrating a probability framework including evidence nodes from diagnostic states of various physiological parameters and one parent node.

FIG. 2 is a block diagram illustrating an example system that includes medical device(s) used to obtain diagnostic states from the various physiological parameters for use as evidence nodes.

FIG. 3 is a block diagram illustrating an example framework for a probability model.

FIG. 4 is a block diagram illustrating an example framework for a probability model that includes a feature extraction model.

FIG. 5 is a conceptual diagram illustrating the environment of an example medical system in conjunction with the patient, including an example implantable medical device (IMD) used to determine physiological parameters of the patient.

FIG. 6 is a block diagram illustrating an example configuration of an IMD.

FIG. 7 is a conceptual side-view diagram illustrating an example IMD.

FIG. 8 is a block diagram illustrating an example configuration of an external device.

FIG. 9 is a flow diagram illustrating an example operation of a system to determine a probability score with respect to a patient, in accordance with one or more techniques disclosed herein.

### DETAILED DESCRIPTION

This disclosure describes techniques for using prediction and probability modeling to classify a health condition and determine a probability score indicating a likelihood of the classification of the health condition being correct. In some examples, the probability score may be based on the application of one or more diagnostic states to the probability model, where the diagnostic states are identified based on physiological parameters acquired from one or more devices (e.g., implanted medical devices, external medical devices, personal devices, etc.).

Processing circuitry of a device may determine respective values for each physiological parameter, and determine the diagnostic states based on the physiological parameter values. Examples of physiological parameters may include or otherwise relate to impedance and temperature. Additional physiological parameters may include blood pressure, heart rate, and one or more parameters indicative of fluid retention. Based on the respective values for the physiological parameters, the processing circuitry may identify one or more diagnostic states defining a plurality of evidence nodes for a probability model. In some examples, impedance values may correspond to a state from a first set of states including a wet state and a dry state, and temperature values may correspond to a state from a second set of states including a cold state and a warm state. In some examples, the first and second set of states may each include any number of states corresponding to different degrees of wetness/dryness and warmness/coldness.

These diagnostic states (e.g., wet state, dry state, cold state, and warm state) may be helpful to a clinician because a clinician may request a patient's HF status by asking whether they're feeling “hot or cold” and “wet or dry.” These questions, which relate to temperature and impedance, may aid clinicians in understanding the patient's current congestion and heart function status and may be used to tailor therapy. Thus, the techniques of this disclosure may help clinicians understand the progression of symptoms as well as aid in the preventative treatment of HF symptoms and decompensation.

The diagnostic states may define a plurality of evidence nodes for a probability model. In some examples, the probability model may represent a deep learning or machine learning model. As an example, the processing circuitry may evaluate impedance values and determine an appropriate state from the first set (e.g., whether the impedance values correspond to a wet state or a dry state). Similarly, the processing circuitry may evaluate temperature values and determine an appropriate state from the second set (e.g., whether the temperature values correspond to a cold state or a warm state). In some examples, the processing circuitry may output the identified diagnostic states to a user, such as a clinician. This may help explain the rationale of classifications rendered by the probability model in accordance with techniques described herein because the user may already be familiar with those diagnostic states when determining a likelihood of a patient experiencing a heart condition.

Diagnostic states of the physiological parameters may be independent for each physiological parameter. For example, a diagnostic state from the first set (e.g., wet) may be independent from a diagnostic state from the second set (e.g., warm). These physiological parameters may independently provide indications of a heart failure event. However, each of these conditionally independent physiological parameters may provide stronger evidence when used together to predict an adverse health event. Accordingly, in some examples, the processing circuitry may determine a joint diagnostic state (e.g., a wet/cold state, a dry/cold state, a wet/warm state, a dry/warm state, etc.) based on multiple physiological parameters, such as impedance and temperature.

In some examples, diagnostic states may include a finite number of potential diagnostic states for each physiological parameter. For example, the diagnostic states may include states of high degree, medium degree, or low degree for each physiological parameter. In some instances, one or more of the physiological parameters can have a different number of potential diagnostic states (e.g., one state, two states, three states, etc.), whereas other physiological parameters may have a greater or fewer number of potential diagnostic states. For example, the first set may have five diagnostic states (very wet, wet, neutral, dry, and very dry), whereas the second set may have less than three diagnostic states (cold and warm). In other examples, diagnostic states may include a continuum or sliding spectrum of diagnostic state values, rather than discrete states.

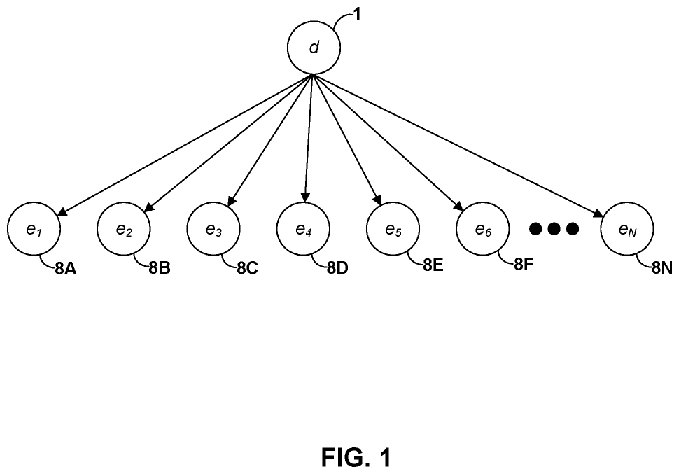

The diagnostic states may serve as evidence nodes for the probability model. FIG. 1 represents an example probability model framework that includes a parent node 1 and a plurality of evidence nodes 8A 8N (collectively, “evidence nodes 8”). Parent node 1 represents the probability of a patient experiencing (e.g., presently or within a predetermined period of time) a health condition based on diagnostic states of evidence nodes 8. In an example, the adverse health event may include a HF event, where d=H, for illustration purposes. The probability model may include any number of evidence nodes 8, as illustrated by evidence node 8N. Each of evidence nodes 8 may correspond to one or more physiological parameters of a patient. As further discussed herein, each one of evidence nodes 8 may include a diagnostic state derived from one or more values for one or more physiological parameters. In examples involving discrete states of d, the probability for the occurrence of the HF event (d=H) may be expressed as:

$$P ( d = H \vert {e}_{1} , … , {e}_{N} ) = \frac{P ( d = H ) {\prod }_{i = 1}^{N} P ( {e}_{i} \vert d = H )}{{\sum }_{{d}_{1}} P ( d ) {\prod }_{i = 1}^{N} P ( {e}_{i} \vert d )}$$

In such examples, P(d) may represent a prior probability value, P(eᵢ|d) may represent a conditional likelihood parameter, d may represent parent node 1, and e₁-e_{N} may represent evidence nodes 8 in FIG. 1. Processing circuitry may determine the prior probability value and the conditional likelihood from existing physiological parameter values prior to clinical event d in previous clinical study data. In some examples, the conditional likelihood parameter may assume, using previous probability data, what probability distribution is likely to exist, such that the processing circuitry can assume what probability scores are unlikely based on previous probability data. In some examples, the prior probability value may include a probability distribution absent any diagnostic states to use as evidence nodes. In other words, the prior probability value is what the processing circuitry may believe at a particular point of time, whereas the probability score is what the processing circuitry may believe in the presence of incoming diagnostic information.

In some examples, the probability score may include a joint probability distribution. In an example, for a n-node Bayesian network (where pai is the parent node of node xi), the joint probability distribution may be expressed as:

$$P ( {x}_{i} , … , {x}_{n} ) = {\prod }_{i}^{N} P ( {x}_{i} \vert {pa}_{i} )$$

For example, determining a probability score may involve determining joint probability distributions and defining multiple combinations of conditional probabilities. A probability model may provide a framework for assumptions regarding the explicit relationship between parameter values to make these determinations more feasible. For example, Bayesian theory may assign explicit relationships between physiological parameter values in order to determine probability scores from the various evidence nodes 8 in FIG. 1.

In some examples, the probability model may output a differential diagnosis rather than a single diagnosis of probability. In other words, the probability model may provide multiple probabilities as output based on the multiple diagnostic parameters. For instance, the probability model may provide, based on evidence nodes 8, a probability of HF, a probability of COPD, probability of infection, probability of sepsis, probability of pneumonia, etc. Each probability of a condition may represent a unique parent node like parent node 1 of FIG. 1.

In some examples, the processing circuitry may determine and/or utilize conditional likelihood tables, BBN tables, prior probability values, etc., in accordance with the techniques described in U.S. application Ser. No. 13/391,376 by Sarkar et al., entitled “METHOD AND APPARATUS FOR MONITORING TISSUE FLUID CONTENT FOR USE IN AN IMPLANTABLE CARDIAC DEVICE,” filed on Feb. 20, 2012, and incorporated herein by reference in its entirety. For example, the conditional likelihood parameters may take the form of conditional likelihood tables defined for each diagnostic state for each physiological parameter. The probability may then be tabulated for all possible combinations of diagnostic states to determine a probability, or in some instances, a probability table, as described in U.S. application Ser. No. 13/391,376.

In some instances, a single evidence node may be derived from multiple physiological parameters, such as with a Multi Variable Node (MVN). In an example, MVNs may be based on multiple physiological parameters, such as impedance as a first physiological parameter and temperature as a second physiological parameter, where the physiological parameters factor into a single evidence node.

The processing circuitry may use the evidence nodes as input to a probability model to determine a probability score. The probability score may indicate a likelihood of a classification of a heart condition of a patient being correct. In some instances, the classification may be a prediction of whether the patient is experiencing an adverse health event or is likely to experience an adverse health event within a predetermined period of time (e.g., within 30 days of determining the probability score). As discussed herein, the probability model may use as additional inputs the prior probability value and a conditional likelihood parameter to determine the probability score. In some examples, the processing circuitry may then update the probability model using the determined probability score.

In some examples, the probability score is compared to one threshold for each of one or more risk levels (e.g., high risk threshold, medium risk threshold, low risk threshold). For example, an alert may be generated when the probability score crosses a first, higher threshold. The alert may end when the probability score subsequently crosses a second, lower threshold. By generating alerts in this manner, a device may generate fewer “sporadic” alerts that may be misinterpreted by the patient or a clinician when the probability score fluctuates near the higher, alert threshold value.

Although primarily described herein with respect to the example probability model framework of FIG. 1, it should be understood that the techniques of this disclosure may be implemented in a variety of ways without departing from the scope of the claims. For instance, in some examples, the prediction and/or probability modeling according to the techniques described herein may include Bayesian Belief Networks (BBN) or Bayesian machine learning (ML) models (these sometimes referred to as Bayesian Networks or Bayesian frameworks herein), Markov random fields, graphical models, artificial intelligence (AI) models (e.g., Naive Bayes classifiers, deep learning neural networks such as convolution neural networks, recurrent neural networks), and/or other belief networks, such as sigmoid belief networks, deep belief networks (DBNs), etc. In other examples, the disclosed technology may leverage non-Bayesian prediction or probability modeling, such as frequentist inference modeling or other statistical models. In addition, known model selection techniques, such as Bayesian information criterion (BIC) or Akaike information criterion (AIC), may be used to evaluate probability models prior to use. In some examples, an integrated diagnostics model may be used to determine a number of criteria that are met based on each physiological parameter.

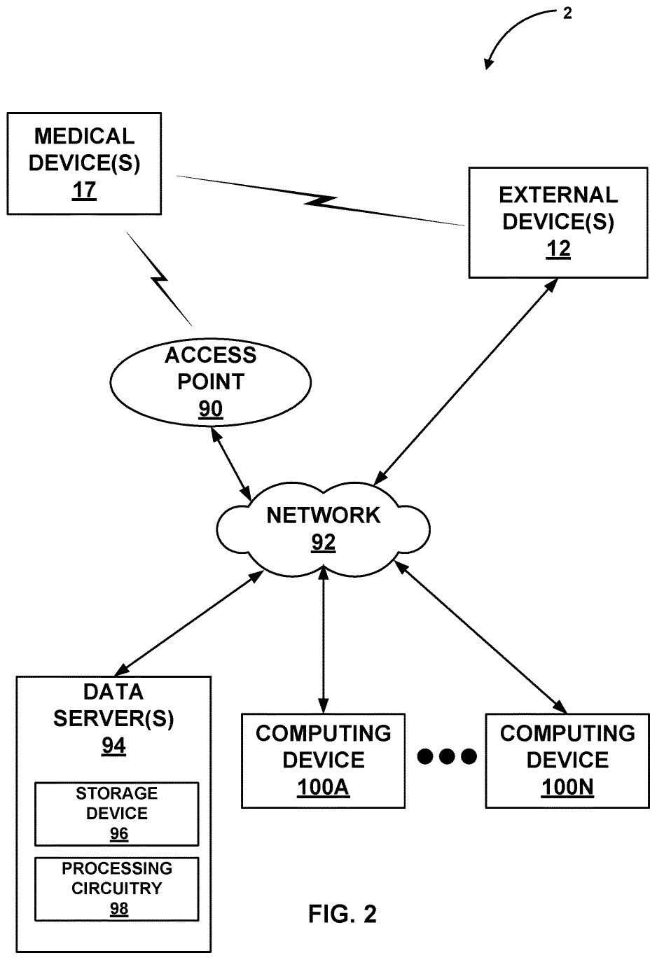

FIG. 2 is a block diagram illustrating an example system 2 that includes one or more external devices 12, one or more medical devices 17, an access point 90, a network 92, one or more data servers 94, and one or more other computing devices 100A-100N (collectively, “computing devices 100”). In some examples, medical devices 17 may include an implantable medical device (IMD). In this example, medical devices 17 may use communication circuitry 54 to communicate with external devices 12 via a first wireless connection, and to communicate with an access point 90 via a second wireless connection.

In one or more of the various example techniques described with reference to FIG. 2, access point 90, external devices 12, data servers 94, and computing devices 100 may be interconnected and may communicate with each other through network 92. Network 92 may include a local area network, wide area network, or global network, such as the Internet. System 2 may be implemented, in some aspects, with general network technology and functionality similar to that provided by the Medtronic CareLink® Network, developed by Medtronic, Inc., of Minneapolis, MN.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient.

Medical devices 17 may include an insertable or implantable medical device and one or more external devices of any type configured to sense or otherwise obtain temperature data as well as other data that may influence physiological parameter values. Other data may include acceleration data, patient-reported data, location data, etc.

Medical devices 17 may be configured to transmit data, such as sensed, measured, and/or determined values of physiological parameters (e.g., impedance measurements, impedance scores, fluid indices, temperature measurements, heart rates, respiratory rate, activity data, cardiac electrograms (EGMs), historical physiological data, blood pressure values, etc.), to access point 90 and/or external device 12. In some examples, medical devices 17 may be configured to determine multiple physiological parameters. For example, medical devices 17 may include an IMD configured to determine subcutaneous tissue impedance values, temperature values, EGM values, respiration rate values, etc. In such examples, the IMD may provide multiple physiological parameters to serve as evidence nodes to probability model 19. A patient's clinical history, lab measurements, and/or measurements from peripheral systems that may be retrieved from electronic medical records (EMR) systems may also serve as evidence nodes to probability model 19. Access point 90 and/or external devices 12 may then communicate the retrieved data to data servers 94 via network 92.

External devices 12 may be a computing device, such as a notebook computer, tablet computer, computer workstation, one or more servers, cellular phone, personal digital assistant, handheld computing device, networked computing device, or another computing device that may implement techniques of this disclosure. In some examples, external devices 12 may be used to retrieve data from medical devices 17. The retrieved data may include impedance values and temperature values measured by medical devices 17, and/or other physiological signals recorded by medical devices 17. For example, external devices 12 may retrieve information related to detection of an increase or decrease in impedance and/or temperature detected by medical devices 17, such as a rate of change that exceeds a predefined threshold.

In the example illustrated by FIG. 2, data servers 94 include a storage device 96 (e.g., to store data retrieved from medical devices 17) and processing circuitry 98. Although not illustrated in FIG. 2 computing devices 100 may similarly include a storage device and processing circuitry. Processing circuitry 98 may include one or more processors that are configured to implement functionality and/or process instructions for execution within data servers 94. For example, processing circuitry 98 may be capable of processing instructions stored in memory 96. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of data servers 94 and/or the processing circuitry of computing devices 100 may implement any of the techniques described herein to analyze physiological parameters received from medical devices 17, e.g., to determine a probability score of patient 4. Furthermore, processing circuitry 98 of data servers 94 and/or the processing circuitry of computing devices 100 may calibrate the physiological parameters received from medical devices 17 using, e.g., measurements obtained by external devices 12.

In some examples, storage device 96 of data servers 94 may store a probability model. In some examples, external devices 12 and/or medical devices 17 may store the probability model. In some examples, data servers 94 may transmit the probability model to one or more of external devices 12 or medical devices 17. External devices 12, medical devices 17, and/or data servers 94 may use the probability model to determine a probability score with respect to a classification of a health condition of a patient.

Processing circuitry 98 of data servers 94 and/or the processing circuitry of computing devices 100 may also retrieve instructions for utilizing a selected probability model (e.g., one selected using a known selection technique) and execute the probability model to determine the probability score. Processing circuitry 98 of data servers 94 and/or the processing circuitry of computing devices 100 may retrieve such data and instructions from storage device 96 or in some instances, from another storage device, such as from one of medical devices 17.

Storage device 96 may include a computer-readable storage medium or computer-readable storage device. In some examples, memory 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

In some examples, system 2 (e.g., one or more of external devices 12, medical devices 17, or data servers 94) may process the physiological parameter values to perform calibration. Calibration may ensure the accuracy of the physiological parameter values. As such, system 2 may perform calibration to convert the physiological parameter values into clinically relevant measurement units.

For instance, external devices 12 may prompt a patient to manually obtain a calibration measurement around a time that medical devices 17 is automatically measuring physiological parameter values. The manual measurements may include metadata (e.g., the time of the manual measurement, the source of the manual measurement, etc.), and system 2 may use the manual measurement to calibrate the temperature measurements collected by medical devices 17. For example, processing circuitry may determine a difference between the manual measurement and a temperature measurement collected by medical devices 17 at about the same time and calibrate (e.g., offset) the temperature measurement collected by medical devices 17 if the difference is excessive (e.g., if the difference exceeds an allowable measurement error threshold). In some examples, system 2 may calibrate the physiological parameter values by using an average (e.g., a mean, median, mode, etc.) of the physiological parameter values to determine diagnostic states, physiological parameter trends, etc. Using a median instead of a mean may remove extreme values from analysis and may be more accurate for that reason.

Additionally or alternatively, medical devices 17 may collect physiological parameter values when the physiological parameter values are relatively stable to reduce or minimize noise. In other words, system 2 may calibrate the physiological parameter values by discarding, not collecting, or otherwise excluding a set of physiological parameter values that fail to satisfy a noise condition from being used to identify the diagnostic states. For example, medical devices 17 may collect temperature data when a patient is brushing his teeth but not when the patient is taking a shower. In some instances, medical devices 17 may collect physiological parameter values based on device orientation, which may indicate, for example, the posture of the patient (e.g., supine, prone, etc.) and in turn whether the patient is sleeping, ambulating, and so on. In some examples, instead of or in addition to medical devices collecting physiological parameter values in this manner, one or more other devices, e.g., external device 12, access point 90, and/or data server(s) 94, may filter or screen out physiological parameter values associated with instability, noise, or certain postures or activities of the patient.

In some examples, physiological parameter values may fail to satisfy the noise condition if the physiological parameter values are below a lower threshold or above an upper threshold, which may indicate that the physiological parameter values deviate so much from the norm that the likelihood of the physiological parameter values being accurate is relatively low. In some examples, the upper and lower thresholds may be predetermined and/or fixed. In other examples, the upper and lower thresholds may be relative to (e.g., a percentage of) recent physiological parameter values. For instance, the upper threshold for temperature may be 105% of the median of the last 30 temperature measurements, and the lower threshold for temperature may be 95% of the median of the last 30 temperature measurements.

In another example, medical devices 17 may collect measurements at the same time-of-day to reduce or prevent the influence of diurnal variations. In yet another example, system 2 may collect measurements based on patient input (e.g., a time when the patient indicates the patient is likely sleeping). For instance, external devices 12 may prompt the patient to complete a questionnaire, in this way soliciting additional context and information that may be used to diagnose the patient and/or calibrate the physiological parameter values. In some examples, the questionnaire may include questions relating to travel, exposure to contagions and/or pathogens, etc.

System 2 may calibrate one or more sensors of medical devices 17 using data from other devices, such as external devices 12. In examples where system 2 calibrates the sensors of medical devices 17 using data from external devices 12, the data from external devices 12 may be collected by sensors of external devices 12 and/or otherwise obtained by external devices 12 (e.g., provided by a patient via an input device of external devices 12, transmitted to external devices 12 via network 92, etc.).

As an example, system 2 may use external devices 12 to sense environmental temperature (or external temperature) data. Additionally or alternatively, system 2 may use location data (e.g., collected by external devices 12) to obtain estimated environmental temperature from a weather database or the like. In any case, processing circuitry may calibrate the temperature data collected by medical devices 17 by removing the influence of the environmental temperature on the temperature data collected by medical devices 17. In general, ambient (or environmental) temperatures that are relatively hot or cold may correspondingly affect (e.g., increase or decrease, respectively) internal temperature values collected by medical devices 17, thus highlighting the importance of calibration in classifying health conditions. Other physiological parameters may be similarly affected by measurements obtained by external devices 12 and consequently benefit from calibration.

In some instances, one or more of medical devices 17 may transmit data over a wired or wireless connection to data servers 94 and/or external devices 12. For example, data servers 94 may receive data from medical devices 17 and/or external devices 12. In another example, external devices 12 may receive data from data servers 94 and/or medical devices 17, such as physiological parameter values, diagnostic states, or probability scores, via network 92. In such examples, external devices 12 may determine the data received from data servers 94 or from medical devices 17 and may store the data accordingly. In another example, external devices 12 may send data to data servers 94 and/or medical devices 17, such as physiological parameter values, diagnostic states, or probability scores, via network 92.

In addition, one or more of medical devices 17 may serve as or include data servers 94. For example, medical devices 17 may include enough storage capacity or processing power to perform the techniques disclosed herein on a single one of medical devices 17 or on a network of medical devices 17 coordinating tasks via network 92 (e.g., over a private or closed network). In some examples, one of medical devices 17 may include at least one of the data servers 94. For example, a portable/bedside patient monitor may be able to serve as a data server, as well as serving as one of medical devices 17 configured to obtain physiological parameter values from a patient. In other examples, data servers 94 may communicate with each of medical devices 17, via a wired or wireless connection, to receive physiological parameter values or diagnostic states from medical devices 17. In a non-limiting example, physiological parameter values may be transferred from medical devices 17 to data servers 94 and/or external device 12.

In some cases, data servers 94 may be configured to provide a secure storage site for data that has been collected from medical devices 17 and/or external device 12. In some instances, data servers 94 may include a database that stores medical- and health related data. For example, data servers 94 may include a cloud server or other remote server that stores data collected from medical devices 17 and/or external device 12. In some cases, data servers 94 may assemble data in web pages or other documents for viewing by trained professionals, such as clinicians. One or more aspects of system 2 may be implemented with general network technology and functionality, which may be similar to that provided by the Medtronic CareLink® Network.

In some examples, system 2 includes one or more of computing devices 100. Computing devices 100 may be a tablet or other smart device located with a clinician, by which the clinician may, for instance, program, receive alerts from, and/or interrogate medical devices 17. For example, the clinician may access data collected by medical devices 17 through a computing device 100, such as when patient 4 is in between clinician visits, to check on a status of a medical condition. In some examples, the clinician may enter instructions for a medical intervention for patient 4 into an application executed by computing device 100, such as based on a status of a patient condition determined by medical devices 17, external devices 12, data servers 94, or any combination thereof, or based on other patient data known to the clinician.

One computing device 100 may transmit instructions for medical intervention to another of computing devices 100 located with a patient or a caregiver of the patient. For example, such instructions for medical intervention may include an instruction to change a drug dosage, timing, or selection, to schedule a visit with the clinician, or to seek medical attention. In further examples, a computing device 100 may generate an alert to the patient (or relay an alert determined by a medical device 17, external device 12, or data sever 94) based on a probability score determined from physiological parameter values of the patient, which may enable the patient to proactively seek medical attention prior to receiving instructions for a medical intervention. In this manner, the patient may be empowered to take action, as needed, to address his or her medical status, which may help improve clinical outcomes for the patient.

In some examples, system 2 may trigger an alert in response to satisfaction of an alert condition. In some examples, the alert condition may be satisfied if the physiological parameter values satisfy a threshold condition for at least a predetermined amount of time. For example, the alert condition may be satisfied if temperature values exceed a threshold value for 24 hours. In some examples, the alert condition may be satisfied if one or more numerical properties of the temperature measurements change. For instance, the alert condition may be satisfied if the maximum temperature measurement increases, the median of the temperature measurements increases, and/or the average of the temperature measurements increases while the variance of the temperature measurements decreases. In general, satisfaction of the alert condition may be based on calibrated or uncalibrated physiological parameter values.

As noted above, system 2 may collect physiological parameter values from one or more sources. Sources may include temperature data measured by medical devices 17, which may be an IMD, impedance and accelerometer data medical devices 17, physiologic data measured by medical devices 17, patient reported data (e.g., temperature, other signs and/or symptoms, etc.) location, temperature, and accelerometer data from external devices 12, and so on. In some examples, system 2 may collect physiological parameters at predetermined times and frequencies. For instance, system 2 may collect physiological parameter measurements at the same time or times of day. As an example, system 2 may collect temperature measurements at 12 a.m., 6 a.m., 12 p.m., and 6 p.m. every day.

System 2 may analyze the set of temperature measurements that are collected at the same time-of-day to determine satisfaction of the alert condition in order to reduce the influence of environmental factors, such as diurnal variations. As an example, system 2 may analyze the set of temperature measurements collected at 6 a.m. to determine satisfaction of the alert condition (e.g., if the median of the temperature measurements has increased). In some examples, system 2 may analyze whether two or more sets of temperature measurements each satisfy one or more alert conditions, where each set of temperature measurements is collected at a different time-of-day. In this way, the two or more sets of temperature measurements may corroborate each other, increasing the likelihood that an alert of an adverse health condition is warranted.

In some examples, system 2 may require confirmation before triggering an alert. For example, responsive to the one or more alert conditions being satisfied, processing circuitry of external devices 12 may output a notification prompting a patient to manually measure the patient's temperature to confirm the temperature measurements collected by medical devices 17. Additionally or alternatively, processing circuitry may evaluate the presence of other physiologic indicators of a health condition, such as increased heart rate. Furthermore, the processing circuitry may evaluate the presence of noise sources, such as patient activity or position (e.g., exercise-induced hyperthermia). As such, the one or more alert conditions may represent a primary criteria for triggering an alert, and confirmation of the physiological parameters and/or the presentation of symptoms may represent secondary criteria. Usage of both the primary criteria and secondary criteria may reduce the occurrence a triggered alert being a false-positive.

In any case, a user of system 2 (e.g., a patient, clinician, etc.) may receive an alert (e.g., via external devices 12, computing devices 100, etc.) indicating changes in the physiological parameters and/or the onset (or imminent onset) of a health condition. For instance, responsive to satisfaction of the alert condition, system 2 may trigger an alert notifying the patient and/or a clinician of recent physiological parameter values and/or the trend in the physiological parameter values.

If measurements collected by medical devices 17 indicate a trend in physiological parameter values (e.g., a general increase in physiological parameter values, a general decrease in physiological parameter values, etc.) of a patient, processing circuitry may trigger an alert notifying a user of system 2 (e.g., a patient, clinician, etc.) of the trend. In some examples, processing circuitry may cause a display to present the physiological parameter values and trends thereof. For instance, the display may present the physiological parameter values collected during a time period (e.g., a day, a week, etc.) as well as trends reflecting patterns and overall changes in the physiological parameter values. Displaying the physiological parameter values and trends in this way may help a patient and/or clinician look for “spikes” in the physiological parameter values and trends, which may be precursor to an adverse health condition. For example, a patient, who may or may not be experiencing symptoms, may look at the trends and take intervening action in response.

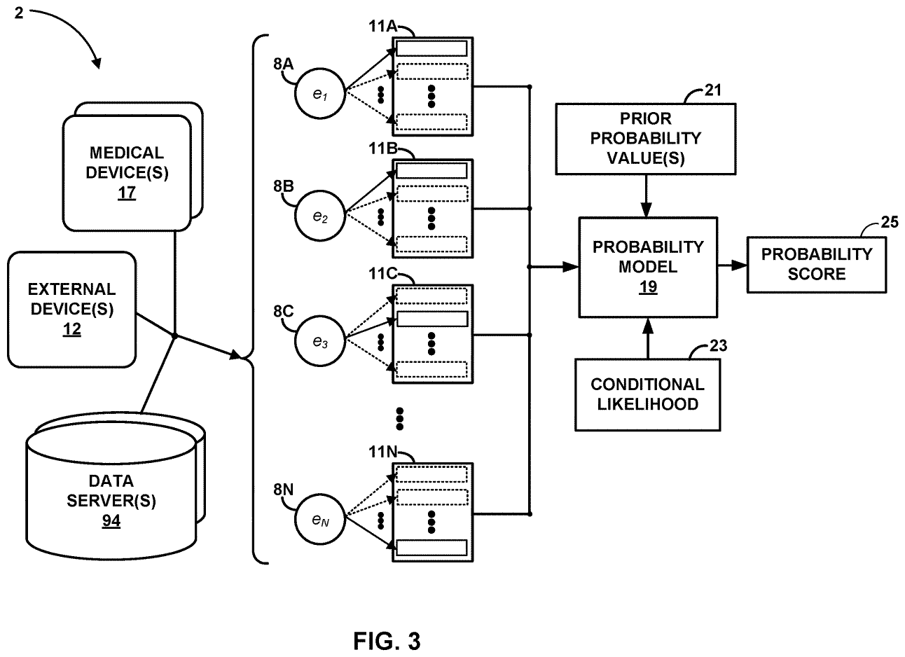

FIG. 3 illustrates a framework that uses system 2 to monitor and classify health events and the likelihood of health events of a patient. System 2 may include one or more external devices 12, one or more medical devices 17, and/or one or more data servers 94. Although primarily described in terms of one or more data server 94 determining the probability score, it should be understood that any one or more devices (e.g., processing circuitry of such devices), such as one or more external devices 12, one or more medical devices 17, and/or other computing devices, may perform the probability determination using a probability model 19 as described herein. In any event, FIG. 3 illustrates external devices 12, medical devices 17, and/or data servers 94 as being configured to supply input to probability model 19.

In some examples, a storage device of data servers 94 (e.g., storage device 96) may store the physiological parameter values that relate to one or more physiological parameters, which may have been received from one or more devices (e.g., one or more external devices 12, one or more medical devices 17, and/or other computing devices) of system 2 via a network. Data servers 94 may store the physiological parameter values as raw data or as calibrated data via calibration techniques (e.g., the calibration techniques described with respect to FIG. 2). For example, data servers 94 may store impedance and temperature values as values determined from subcutaneous tissue impedance data and temperature data collected by medical devices 17 and external devices 12.

Processing circuitry, e.g., processing circuitry of data servers 94, may store data received from external devices 12 and/or medical devices 17 to a storage device, e.g., storage device 96 of data servers 94. In an example, storage device 96 may be configured to store measured and/or determined values of one or more impedance parameters and temperature parameters. The one or more impedance parameters may include subcutaneous tissue impedance values or scores, fluid index values, etc. The one or more temperature parameters may include core body temperature values, surface temperature values, external temperature values, etc.

In some examples, data server 94 may receive physiological parameter values (e.g., raw or calibrated data) from medical devices 17 via network 92. In such examples, processing circuitry 98 of data servers 94 may determine an index or score values used to determine inputs to probability model 19. In some examples, medical devices 17 or external devices 12 or another device may determine the index or score values from one or more subcutaneous tissue impedance measurements. Medical devices 17 may determine one or more subcutaneous tissue impedance measurements via one or more electrodes. In some examples, subcutaneous tissue impedance measurements via one or more electrodes may help distinguish a pocket infection from a general fever. For instance, electrode impedance may detect evidence of local edema or bulk changes in tissue properties due to a pocket infection.

Processing circuitry 98 of data servers 94 may determine one or more impedance scores using impedance value measurements. Processing circuitry 98 of data servers 94 may determine one or more temperature scores using temperature value measurements. In some examples, data servers 94 may receive one or more subcutaneous tissue impedance scores and temperature scores, where another device, such as external devices 12, may determine the subcutaneous tissue impedance scores and temperature scores prior to transmitting the impedance scores and temperature scores to data servers 94. In any case, data servers 94 may receive data from external devices 12 and/or medical devices 17 and determine, via probability model 19, a probability score based on the data.

With reference still to FIG. 3, processing circuitry 98 of data servers 94 (or processing circuitry of any other device of the system) may, in some examples, perform the probability score determination using probability model 19 in accordance with the following. As described herein, processing circuitry 98 may be coupled to one or more storage devices such that processing circuitry 98 may leverage the various data repositories in order to determine the probability score.

In some examples, processing circuitry 98 may be configured to determine a respective one or more values for each of a plurality of physiological parameters. For instance, processing circuitry may determine one or more values for a first physiological parameter (e.g., impedance) and one or more values for a second physiological parameter (e.g., temperature). In one example, the values may correspond to measurement readings determined via medical devices 17. For example, the values may include impedance values, temperature values, respiration rate values, ECG values, activity level values, etc. In some examples, the values may include impedance values that indicate fluid retention. The values may include temperature values that indicate changes in internal body temperature. The values may indicate when the patient was active or inactive. The values may include accelerometer values that indicate a posture of the patient or a change in the posture of the patient over time (e.g., a posture-change count). The posture-change count may be based on z-axis accelerometer values. Other values may include periodic x, y, and z-axis accelerometer measurements.

As described herein, the plurality of physiological parameters may include one or more subcutaneous tissue impedance parameters identified from the one or more subcutaneous tissue impedance measurements. The one or more subcutaneous tissue impedance parameters may include a subcutaneous tissue impedance score, as well as fluid index values. The subcutaneous tissue impedance score may be determined in accordance with techniques described in a commonly assigned and co-pending applications by Sarkar et al., entitled “DETERMINING HEART CONDITION STATUSES USING SUBCUTANEOUS IMPEDANCE MEASUREMENTS,” and “DETERMINING HEALTH CONDITION STATUSES USING SUBCUTANEOUS IMPEDANCE MEASUREMENTS” filed on Sep. 27, 2019, and incorporated herein by reference in their entirety. In other examples, the subcutaneous tissue impedance parameters may include tissue perfusion measurements, fluid index values, statistical representations of subcutaneous tissue impedance measurements, respiration rate, etc. In some examples, fluid index values may be derived from other sensors, such as intra-cardiac pressure sensors. For example, an intra-cardiac pressure sensor may detect higher pressures, which may be indicative of a higher amount of fluid. The cardiac pressure data may be used to compute one or more fluid index values and/or scores based on fluid index values. In some examples, tissue perfusion measurements may be derived from optical sensors.

In some examples, processing circuitry 98 may be configured to identify diagnostic states 11A-11N (collectively, “diagnostic states 11”) for each of the physiological parameters based on the respective values. For example, various thresholds may be used to determine a diagnostic state of a physiological parameter. In some examples, processing circuitry 80 is configured to select a single diagnostic state for each of evidence nodes 8. The diagnostic states may be selected from N number of diagnostic states. For example, diagnostic state 11A may be a first diagnostic state (e.g., wet), diagnostic state 11B may be a second diagnostic state (e.g., warm). Diagnostic states 11, independently or jointly, may be associated with a risk level or risk categorization (e.g., low risk, medium risk, high risk, etc.). For instance, a joint diagnostic state of dry/warm may be associated with low risk; a joint diagnostic state of dry/cold may be associated with medium risk; a joint diagnostic state of wet/warm may be associated with medium risk; and a joint diagnostic state of wet/cold may be associated with high risk. A joint diagnostic state of wet/cold may be associated with high risk because patients with a relatively low body temperature and who have high fluid levels may be at increased mortality risk.

In general, a joint diagnostic state of wet/cold is directly related to HF, indicating a very high probability of HF. A joint diagnostic state of wet/warm is likely related to HF, indicating a high probability of HF. Wet/warm may also be related to infection or sepsis. A joint diagnostic state of dry/cold may be related to HF but may be related to other conditions. A joint diagnostic state of dry/warm may be related to infection or sepsis.

An upward trend in temperature may indicate infection and/or fever, and a downward trend in temperature may indicate lack of perfusion or cardiovascular shock. To be more specific, if temperature increases (e.g., “warm”) and there is more fluid retention (e.g., “wet”), then the probability is high that the patient is experiencing an alternate precipitating condition such as pneumonia or COPD which is leading to fluid retention with HF being a secondary cause. If the temperature increases and there is no increase in fluid retention, the patient is likely experiencing an infection (e.g., respiratory, viral, or bacterial). If temperature decreases and fluid retention increases, decreases, or remains the same, then these trends may signify lack of perfusion or vasoconstriction or reduction in cardiac output.

In some examples, probability model 19 may output a differential diagnosis based on evidence nodes 8. For example, probability model 19 may provide multiple probabilities as output based on the multiple diagnostic parameters, such as a probability of HF, a probability of COPD, probability of infection, probability of sepsis, probability of pneumonia, etc.

Diagnostic states 11 may be determined independently for each physiological parameter (e.g., impedance, temperature, etc.). For example, processing circuitry 98 may compare the values obtained for a first physiological parameter to one or more thresholds to determine a diagnostic state for the first physiological parameter independently of processing circuitry 98 comparing values obtained for a second physiological parameter to one or more thresholds to determine a diagnostic state for the second physiological parameter. In some examples, data servers 94 may receive diagnostic states 11. In other examples, data servers 94 may determine diagnostic states 11 based on the respective values of the physiological parameters. In any event, diagnostic states 11 may define evidence nodes 8 for probability model 19. In other words, diagnostic states 11 may serve as evidence nodes 8 for probability model 19.

In some examples, the physiological parameters may additionally include heart rate metrics, such as heart rate or R-R interval, heart rate variability (HRV) or night heart rate (NHR), activity, or a quantification of atrial fibrillation (AF) or other arrhythmia experienced by the patient. Moreover, the physiological parameters may include posture, respiratory effort, R-wave amplitude or other ECG morphological measurements, heart sound, nighttime rest versus daytime active body angle, chronotropic incompetence, B-type natriuretic peptide (BNP), renal dysfunction (e.g., Creatinine or Potassium), blood pressure, blood glucose, etc. In addition, physiological parameters may include posture-change count and accelerometer data values. In any event, the physiological parameters may include impedance parameters and temperature parameters.

In some examples, probability model 19 may include a Bayesian framework or BBN. Other suitable probability models may be used to determine probability scores given diagnostic states of physiological parameters. For example, a Bayesian ML model may be used to determine probability scores based on diagnostic states of physiological parameters. A computing system (e.g., processing circuitry 98 of data servers 94) may train probability model 19 on values associated with the physiological parameters. As shown in FIG. 3, probability model 19 receives as input prior probability values 21 and conditional likelihood 23. Processing circuitry 98 may determine, from the plurality of physiological parameters, prior probability value 21. The prior probability value 21 may be determined from existing data. Processing circuitry 98 may also determine, from the plurality of physiological parameters, conditional likelihood parameter 23.

In some examples, processing circuitry 98 may determine the conditional likelihood, or P(eᵢ|d), from existing data. Processing circuitry 98 may utilize existing data from one or more patients or subjects, where the existing data is then used to determine conditional likelihood parameters of a model utilizing a probability theorem, such as Bayes rule.

In one example, the value of d may represent the presence or absence of an HF event or other adverse health event. As such, processing circuitry 98 may use earlier data to determine whether a particular diagnostic criterion was satisfied before an HF event (e.g., P(eᵢ|d=1)) or whether the particular diagnostic criterion was satisfied when there was no HF event (e.g., P(eᵢ|d=0)). In an example, using a first evidence node e₁ as corresponding to ‘impedance score’, processing circuitry 98 may determine from a plurality of existing data points the conditional likelihood for: P(e₁=H|d=1), P(e₁=H|d=0), P(e₁=M|d=1), P(e₁=M|d=0), P(e₁=L|d=1), and P(e₁=L|d=0). That is, processing circuitry 98 may determine the conditional likelihood from data derived from True Positives, False Positives, False Negatives, and False Positives. In some examples, processing circuitry 98 may use the same data to provide a desired sensitivity and specificity of HF detection. In some examples, processing circuitry 98 use the same data to provide a desired sensitivity and specificity of HF detection. That is, probability 25 may represent an estimate of positive predictive value (PPV) based on sensitivity, specificity and event rate (e.g., prior probability 21).

Processing circuitry 98 may determine the conditional likelihoods for each physiological parameter used as an input evidence node to probability model 19 (e.g., each of eᵢ). In some examples, processing circuitry 98 may then utilize the conditional likelihood probabilities to determine the probability model 19. In such examples, the determined probability model 19 may include a computable joint distribution.

As such, processing circuitry 98 may identify prior probability value 21 and/or the conditional likelihood parameter 23 as inputs to probability model 19 when determining the probability score. In such examples, the probability model may be expressed as:

$$P ( d , {e}_{1} , … , {e}_{N} ) = P ( d ) {\prod }_{i = 1}^{N} P ( {e}_{i} \vert d )$$

where P(d) represents the prior probability value, P(eᵢ|d) represents the conditional likelihood parameter, d represents a parent node, and e₁-e_{N} represent the evidence nodes.

In some examples, processing circuitry 98 may be configured to determine a probability score from probability model 19 based on evidence nodes 8. In an example, the BBN may have one or more child nodes (e.g., n-nodes) and a parent node, represented by posterior probability 25.

The probability score may include a likelihood that the patient is experiencing an adverse health event or is likely to experience the adverse health event within a predetermined amount of time. In an example, the adverse health event could be a worsening HF event (e.g., HF decompensation). The probability score may be expressed in terms of a percentage, a decimal number, or a threshold categorization, such as 50%, 0.5, or medium likelihood, where in this example, 50% corresponds to a threshold categorization of medium likelihood. In some examples, the probability score may be expressed in terms of a range such as >50% or between 50-60%.

In some examples, processing circuitry 98 may determine the probability score for a predetermined amount of time in the future. This may be known as a look-forward period. In some examples, the predetermined amount of time is approximately 30 days relative to when the probability score is determined. For example, the probability score may indicate that patient 4 has a 50% chance of experiencing an adverse health event in the next 30 days. In some instances, the predetermined amount of time may be more or less than 30 days depending on the particular configuration of probability model 19. As such, probability model may determine a probability score that indicates the likelihood of an adverse health event, such as a heart failure worsening event, occurring within the predetermined timeframe (e.g., next 30 days).

Processing circuitry 98 may, in some instances, determine the predetermined amount of time, such that the predetermined amount of time serves as a buffer period. In other words, at the end of the predetermined amount of time (e.g., 30 days), processing circuitry 98 may determine another probability score using data received during a preceding timeframe (e.g., the last 30 or 60 days). Processing circuitry 98 may perform automatic probability determinations using probability model 19 after the predetermined amount of time and after each buffer period thereafter. In other examples, processing circuitry 98 may determine a probability score in response to receiving a command signal (e.g., from a user via a user interface). Processing circuitry 98 may alter the predetermined timeframe slightly to account for the different days in a month. For example, approximately 30 days may include 31 days, 29 days, or 28 days, for convenience of patient 4. That is, patient 4 may have an easier time tracking buffer periods based on months or weeks rather than based on strict 30-day periods.

In some examples, processing circuitry of system 2 (e.g., processing circuitry 98) may determine the probability score on a daily basis. For example, processing circuitry 98 may determine the probability score every day based on data corresponding to a previous X number of days. In some examples, processing circuitry 98 may store in storage device 96 diagnostic states for various parameters each day for a finite number of days, such as in a first in, first out (FIFO) buffer or sliding window. In some examples, processing circuitry 98 may store the last 30 diagnostic states for each parameter determined on a daily basis for the past 30 days. For example, processing circuitry 98 may store the last 30 diagnostic states for impedance scores determined on a daily basis for the past 30 days, store the last 30 diagnostic states for RR determined on a daily basis for the past 30 days, etc. Processing circuitry 98 may determine the probability score at a predefined time interval each day using the previous 30 diagnostic states of each parameter determined over the past 30 days as input to the probability model 19. In another example, processing circuitry 98 may determine the probability score at a predefined time interval each day using the previous 15 diagnostic states of each parameter determined over the past 15 days as input to the probability model 19. In any event, processing circuitry 98 may receive data from medical devices 17 on a periodic basis, such as on a daily, weekly, or biweekly basis, etc. In such examples, processing circuitry 98 may determine the probability score responsive to receiving the data from medical devices 17 according to the periodic transmission rate of medical devices 17 (e.g., daily, weekly, biweekly, etc.). In one example, processing circuitry 98 may determine diagnostic states (e.g., risk states) for each physiological parameter. In such examples, processing circuitry 98 may combine the last X number of days of diagnostic states together to determine a probability score using probability model 19.

In another example, processing circuitry 98 may determine the probability score (e.g., risk score) and diagnostic states on a periodic basis. In addition, processing circuitry 98 may determine the status of the health condition of patient 4 using the probability score and a threshold on a periodic basis. In a non-limiting example, processing circuitry 98 may compute the probability score, diagnostic states, and status on a daily basis. In such examples, processing circuitry 98 may store the probability score and/or diagnostic state for the last X number of days, such as for the last 30 days. In some examples, processing circuitry 98 may determine the probability score on a day basis using diagnostic data from the past X number of days, such as the last 30 days. In such examples, processing circuitry 98 may determine, on any given day, that the probability score satisfies a threshold. For example, processing circuitry 98 may determine that the probability score exceeds a threshold. In such examples where processing circuitry 98 determines that probability score satisfies a threshold, processing circuitry 98 may transmit an alert externally, such as to a physician device or patient device.

Although described with reference to processing circuitry 98, the techniques of this disclosure are not so limited. In some examples, other processing circuitry (e.g., processing circuitry of one or more of external devices 12, processing circuitry of one or more of medical devices 17, such as a central processing unit (CPU) of one of medical devices 17, etc.) may perform one or more of the techniques of this disclosure and may coordinate with other devices accordingly. For example, processing circuitry of one of medical devices 17 may determine the probability score on a daily basis, compare the probability score to a threshold, and cause the transmission of an alert where the probability score satisfies the threshold. In such examples, a particular medical device of medical devices 17 may receive data (e.g., diagnostic data) from network 92, such as from other medical devices 17, external device 12, or data servers 94, and may determine the probability score using processing circuitry included with the particular medical device.

With reference still to FIG. 3, processing circuitry 98 may be configured to identify, from the respective one or more values for each physiological parameter, a plurality of physiological parameter features that encode amplitude, out-of-normal range values, and temporal changes. In the example of amplitude, a physiological parameter feature may encode R-wave amplitudes, accelerometer signal amplitudes, etc. For example, processing circuitry 98 may determine whether a particular physiological parameter satisfies an absolute threshold. In an illustrative example, processing circuitry 98 may determine whether an average NHR of a patient is greater than a predefined threshold of 90 bpm. In the example of out-of-normal values, a physiological parameter feature may encode an expected range values to determine whether a physiological parameter includes out-of-normal values to encode. For example, processing circuitry 98 may determine a high heart rate based on expected heart rate values.

In an illustrative example, processing circuitry 98 may determine NHR out-of-range values by comparing the average NHR to determine how many data NHR has been greater than 90 bpm or less than 55 bpm. In the example of temporal changes, a physiological parameter feature may encode changes in a physiological parameter over time. In one example, processing circuitry 98 may encode a feature of subcutaneous impedance measurements with changes in impedance over a period of days or weeks. Similar to calculating the fluid index using impedance values, processing circuitry 98 may determine relative changes in a physiological parameter value to determine temporal changes, rather than absolute changes. In an illustrative example, processing circuitry 98 may determine whether an average or current-day temperature value has increased in a sustained manner over the last 7 days or 30 days relative to temperature values in the last 7 days or 30 days. In such examples, processing circuitry 98 is configured to identify the evidence nodes based at least in part on the plurality of physiological parameter features. For example, processing circuitry 98 may extract features that encode information regarding out-of-normal range values, as well as temporal changes at weekly and monthly time scale for the physiological parameters.

Processing circuitry 98 may extract features from the physiological parameters and/or from the physiological parameter values. For example, processing circuitry 98 may analyze a large set of time series data for each physiological parameter for time windows including the number of days the values are outside a normal amplitude range, cumulative sum of difference between the raw measurement and an adaptive reference (CSAR), cumulative sum of difference between the raw measurement in a fixed reference (CSFR), number of days CSAR or CSFR were above a threshold, slope or rate of change of raw measurement values, or mean, median, minimum, and maximum measurement values. Processing circuitry 80 may extract such features for each physiological parameter to encode amplitude and temporal characteristics with respect to particular temporal scales.

In some examples, processing circuitry 98 (or other processing circuitry described herein) may apply a machine-learning model to extract features from the physiological parameter values. Example feature extraction techniques may include edge detection, corner detection, blob detection, ridge detection, scale-invariant feature transform, motion detection, optical flow, Hough transform, etc. The extracted features can include or be derived from transformations of the input data (e.g., the physiological parameter data) into other domains and/or dimensions. As an example, the extracted features can include or be derived from transformations of the input data into the frequency domain. For example, wavelet transformations and/or fast Fourier transforms can be performed on the input data to generate additional features.

In some examples, the extracted features can include statistics calculated from the input data or certain portions or dimensions of the input data. Example statistics include the mode, mean, median, maximum, minimum, or other metrics of the input data or portions thereof.

In some examples, dimensionality reduction techniques can be applied to the input data prior to input into probability model 19. Several examples of dimensionality reduction techniques include principal component analysis, independent component analysis, kernel principal component analysis, graph-based kernel principal component analysis, principal component regression, partial least squares regression, Sammon mapping, multidimensional scaling, projection pursuit, linear discriminant analysis, mixture discriminant analysis, quadratic discriminant analysis, generalized discriminant analysis, flexible discriminant analysis, autoencoding, etc. For instance, processing circuitry 98 may be configured to calculate eigenvectors and eigenvalues of the input data that help define a coordinate system that optimally describes variance of the input data.

In some examples, processing circuitry 98 may determine a MVN as one of the evidence nodes. For instance, multiple physiological parameters may factor into determining a single child node of evidence nodes 8. In a non-limiting example, processing circuitry 98 is configured to determine an input to a first child node of evidence nodes 8 based on a combination of one or more values. For example, evidence node 8A may be based on a combination of an indication of atrial fibrillation (AF) extent in a patient during a time period and one or more values indicating a ventricular rate during the time period (e.g., during AF). In addition, processing circuitry 98 may be configured to determine an input to evidence node 8B based on the respective one or more values of the one or more subcutaneous tissue impedance parameters. Furthermore, processing circuitry 98 may be configured to determine an input to evidence node 8C based on the respective one or more values of the temperature parameter. In such instances, evidence node 8A may include a combination of an AF extent indication values and ventricular rate values, whereas evidence node 8B may indicate one or more subcutaneous tissue impedance parameter values (e.g., subcutaneous tissue impedance value or score, fluid indices, etc.), and evidence node 8C may indicate one or more temperature parameter values (e.g., core body temperature values, surface temperature values, external temperature values, etc.).

With reference still to FIG. 3, processing circuitry 98 may determine, for each of the plurality of physiological parameters or evidence nodes 8, the respective one or more parameter values at various frequencies. For example, processing circuitry 98 may determine the values for evidence node 8A at a different frequency than for evidence node 8B. Thus, diagnostic states 11 may update at different frequencies. In such examples, processing circuitry 98 may delay execution of probability model 19 until an appropriate number of diagnostic states are deemed current or updated. In any event, processing circuitry 80 may determine the diagnostic states using the respective one or more values. Processing circuitry 98 may use the diagnostic states to determine probability score 25. Processing circuitry 98 may then store, the respective one or more values and/or probability score 25 to, for example, storage device 96.

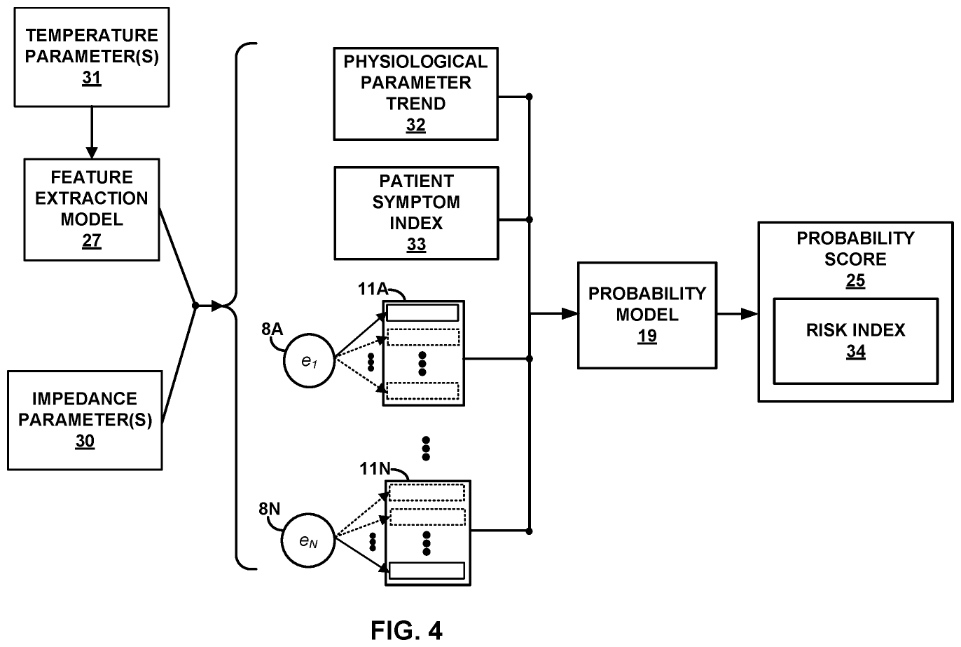

FIG. 4 is a block diagram illustrating an example framework for probability model 19 that includes a feature extraction model 27. Feature extraction model 27 may calibrate physiological parameters, including impedance parameters 30 and/or temperature parameters 31, that probability model 19 receives as input. For example, feature extraction model 27 may apply dimensionality reduction techniques such as principal component analysis. Feature extraction model 27 may be configured to perform dimensionality reduction techniques to determine (e.g., quantify) the effect of various factors, including environmental factors, diurnal variations, etc., that may cause an amount of noise in the temperature values. Responsive to determining the effects of the various factors, processing circuitry 98 may calibrate temperature parameters 31 to remove the effects of the various factors. In some examples, processing circuitry 98 may receive temperature values from another computing system (which may include one or more computing devices) and/or temperature sensing device (e.g., an external thermometer). Feature extraction model 27 may use the temperature values from the computing system and/or temperature sensing device when performing the dimensionality reduction techniques.

Calibration of, for example, temperature parameters 31 may decrease an amount of noise in the temperature values caused by various factors, including environmental factors, diurnal variations, etc. For example, relatively hot or cold ambient temperatures (e.g., due to geography and the time of year) may affect the temperature of a patient and thus internal temperature measurements. In addition, diurnal variations may be attributable to an amount of physical activity of the patient, certain attire worn by the patient (e.g., a jacket worn by the patient in response to cold weather) that increases the temperature of a patient, etc.

In addition to (or alternatively), processing circuitry, e.g., processing circuitry 98 of server 94 (or other processing circuitry described herein) may apply a low pass filter to temperature parameters 31 to calibrate temperature parameters 31. For example, processing circuitry 98 may smoothen the temperature values using a digital filter or in some instances, an analog filter. In one example, processing circuitry 98 may apply a digital filter that increases signal-to-noise ratio (SNR) to create a smoothened temperature signal by filtering out high frequency noise or other high frequency variations from temperature values determined over time. In another example, processing circuitry 98 may smoothen the temperature values using a low pass differentiator filter that performs smoothing based on predefined coefficients and/or smoothing differentiator filter functions to remove high frequency variations in temperature values determined over time.

In some examples, processing circuitry 98 may apply a low pass filter that passes low-frequency temperature variations while impeding high-frequency temperature variations. The low pass filter may have a predefined cutoff frequency that attenuates temperature variations exceeding that of the cutoff frequency. In this way, processing circuitry 98 may apply the filter so as to filter, smooth, or otherwise take into account normal daily variations in temperature values. In some examples, a low pass filter, such as a moving average filter, or other smoothing filter, may be applied to remove normal variations that occur in temperature on a day-to-day basis. For example, in any given day, temperature values may increase during parts of the day when the ambient temperature is increasing or when a person is active and decrease during parts of the day when the ambient temperature is decreasing.

By applying a filter, processing circuitry 98 may attribute less weight to normal variations in daily temperature values, in this way calibrating temperature parameters 31. This is because high-frequency variations tend to be consistent day-to-day, but the actual amplitude of low frequency temperature values may still vary over time, for example, in response to an adverse health condition of a patient. As such, a low pass filter may filter out high-frequency variations from the overall temperature values while allowing low frequency variations to still appear.

In some examples, processing circuitry 98 of server 94 may determine a moving average of temperature parameters 31 over time to calibrate temperature parameters 31. In some examples, processing circuitry 98 may employ a moving average filter. The moving average may be based on a resolution parameter, such that the moving average is determined based on a resolution of daily, bidaily, hourly, etc. That is, processing circuitry 98 may compute the moving average on an hourly basis, daily basis, etc. In some examples, the moving average may be a moving median to exclude outliers.

In some examples, processing circuitry 98 may determine the moving average of temperature values on an hourly basis, daily basis, etc., based on an average of temperature values from the week, month, or other arbitrary period of time prior to a current moving average determination. In a non-limiting example, processing circuitry 98 may determine the moving average of temperature values on day 10 by determining the average of the temperature values from the previous 10 days (days 1-10). In addition, processing circuitry 98 may determine the moving average of temperature values on day 11 by determining the average over the same period of time (days 2-11). In this way, processing circuitry 98 may determine the moving average based on a first-in, first-out (FIFO) buffer that stores finite amounts of temperature data over time (e.g., an X-day FIFO buffer). For example, the FIFO buffer may be a 10-day FIFO buffer that stores temperature values, average temperature values, moving averages of temperature values, etc. for 10 days at a time. In some examples, the moving average may be based on a plurality of moving averages determined over time. For example, a moving average on day 11 may be the sum of A2+A3+ . . . +A11 divided by 11, where A represents the average for the time period denoted by the subscript. For example, A1 may be the average temperature for day 1. A2 may be the average temperature value over days 1 and 2 or in some instances, A2 may be determined based on A1 and the average temperature from day 2. In another example, processing circuitry 98 may determine the moving average using exponential moving averages (EMA) or an otherwise weighted moving average (WMA).

In another example where the resolution comprises determining the moving average on a daily basis, processing circuitry 98 may determine the moving average on day 30 as the average of temperature values measured for the past 30 days and may determine the moving average on day 31 as the average or moving average of temperature values measured for the past 31 days. As such, moving average filters may be used to create a constantly updated average temperature. In this way, a moving average filter may define a current directional trend for a set of temperature values. In any event, probability model 19 may perform more accurately to detect health conditions when physiological parameter values are calibrated using various calibration techniques, including determination of an average (e.g., a mean, median, mode, etc.), exclusion of values that fail to satisfy a noise condition, application of a feature extraction model 27, applying signal processing techniques, and so on.

In some examples, medical devices 17 may be an IMD, and at least one temperature sensor may be located within or fixed to the IMD. In some instances, one or more temperature sensing devices may also be located in or on other parts of the body of a patient, such as with another IMD or external device in communication via network 92. A temperature sensing device may be of a different type or of the same type as the temperature sensor of the IMD. In some examples, the other temperature sensing device may be configured to measure core body temperature. In such examples, processing circuitry 98 may determine the plurality of temperature values over time based at least in part on temperature measurements from each of the temperature sensors and temperature sensing devices. For example, processing circuitry 98 may determine the plurality of temperature values over time based at least in part on temperature measurements from each of the temperature sensors included within the IMD and at least in part on temperature measurements from other temperature sensing devices located in or on other parts of the body of the patient. For instance, the temperature values may be determined from one or more of subcutaneous tissue temperature measurements, endocardial tissue temperature measurements, or intramuscular tissue temperature measurements.

In this way, temperature measurements from multiple temperature sensors may be used to determine whether a health event has occurred in accordance with techniques of this disclosure. Processing circuitry 98 may use the various temperature measurements to determine temperature parameters 31, such as a core body temperature, an IMD temperature, an external temperature, etc. Processing circuitry 98 may calibrate temperature parameters 31 as described above, and probability model may use the calibrated temperature parameters 31 as evidence nodes 8 for determining diagnostic states 11.

In some examples, probability model 19 may receive physiological parameter trend 32 and patient symptom index 33 as input. Physiological parameter trend 32 may describe or otherwise indicate trends over time in physiological parameters, such as impedance parameters 30 and temperature parameters 31. For instance, physiological parameter trend 32 may indicate whether one or more of impedance parameters 30 increased or decreased over a period of time (e.g., 7 days) and/or whether one or more of temperature parameters 31 increased or decreased over a period of time. In some examples, physiological parameter trend 32 may indicate whether physiological parameter measurements collected at specific times (e.g., every day at 12 a.m., 6 a.m., 12 p.m., 6 p.m., etc.), frequencies, and/or intervals increased or decreased. In this way, physiological parameter trend 32 may reflect changes in physiological parameters that are not influenced by diurnal variations (e.g., because the measurements on which the trend is based are collected at the same time of day).

Processing circuitry 98 may determine patient symptom index 33 in a similar manner to calculating the fluid index. That is, processing circuitry 98 may change a patient symptom index value based on a comparison of a current value or short-term average value of a physiological parameter to a baseline or long-term average value of the physiological parameter. The index value may represent short term deviations from the trend that may indicate a health condition. In some examples, the physiological parameter(s) used to determine one or more symptom indices may be the same used to determine diagnostic states, such as warm/cold and wet/dry states.

Probability model 19 may output probability score 25 based on diagnostic states 11. In some examples, probability model 19 may categorize a level of risk that the classification poses to the patient based on the probability score. The level of risk may be a risk index 34. In some examples, risk index 34 may include discrete categories, such as low risk, medium risk, high risk, etc. In other examples, risk index 34 may include a continuum or sliding spectrum instead of discrete categories. In some examples, processing circuitry 98 may determine risk index 34 based on a comparison on probability score 25 to one or more threshold probability levels. In examples where probability model 19 outputs a differential diagnosis and provides multiple probabilities as output, processing circuitry 98 may determine risk index 34 for each of the various diagnoses (e.g., sepsis, infection, COPD, HF, etc.).

As a non-limiting example, diagnostic state 11A may be dry and diagnostic state 11B may be warm. Based on diagnostic states 11A-11B, probability model 19 may output a classification of normal health and a relatively high probability score (e.g., indicating a high probability that the classification of normal health is correct). Accordingly, probability model 19 may output a risk categorization of low risk in accordance with risk index 34. As another diagnostic state 11A may be wet and diagnostic state 11B may be cold. Based on diagnostic states 11A-11B, probability model 19 may output a classification of (imminent) heart failure and a relatively high probability score (e.g., indicating a high probability that the classification of heart failure is correct). Accordingly, probability model 19 may output a risk categorization of high risk in accordance with risk index 34. probability model 19 may output other degrees of risk (e.g., low risk, medium risk, etc.) in accordance with diagnostic states 11.

In some examples, system 2 may trigger an alert in response to determinations by probability model 19. For instance, system 2 may alert a patient, e.g., via a smartphone or other computing device of the patient, if physiological parameter trend 32 indicates changes in temperature and/or impedance. The changes in temperature and/or impedance measurements may be with respect to a maximum value, a median value, etc. In some cases, system 2 may alert a patient if physiological parameter trend 32 indicates changes in temperature and/or impedance and if variance in the physiological parameters measurements has decreased. Alerting a patient of a trend for one or more physiological parameters when variance in the physiological parameters measurements has decreased may result in fewer false classifications (e.g., due to noise in impedance and/or temperature signals) of changes in the physiological parameters, which may increase the reliability of the alerts and in turn the willingness of a patient to respond to the alerts (e.g., by visiting a healthcare professional, such as a clinician).

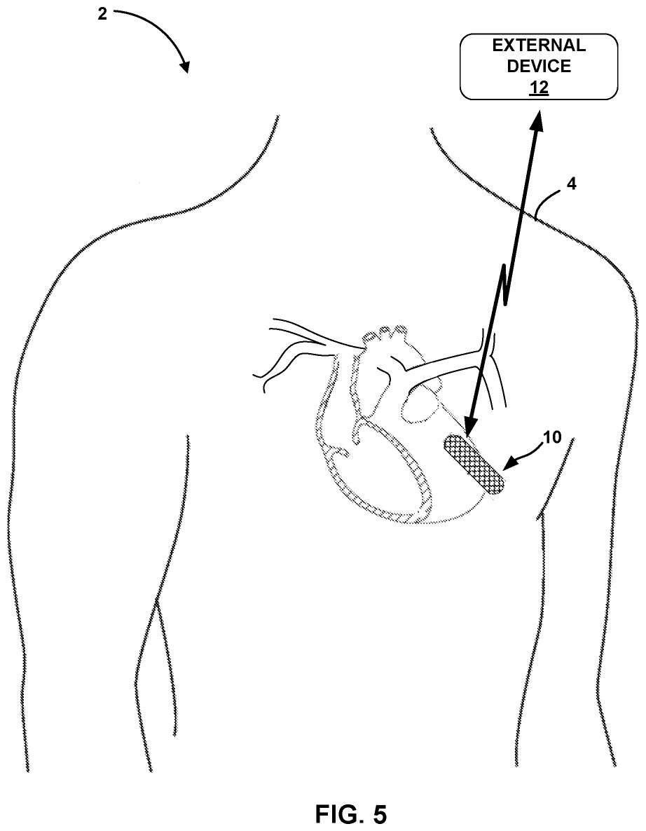

FIG. 5 illustrates the environment of system 2 in conjunction with a patient 4, in accordance with one or more techniques of this disclosure. Patient 4 ordinarily, but not necessarily, is a human. For example, patient 4 may be an animal needing ongoing monitoring for cardiac conditions.

In some examples, system 2 may include an IMD 10. IMD 10 is an example of a medical device 17. In other examples, system 2 may not include IMD 10 and may instead include other medical devices 17 (not shown in FIG. 5), such as a patch monitor, a wearable cardioverter defibrillator (WCD), etc. In any case, any of the various examples of medical devices 17 may be configured in accordance with the techniques in a similar manner as IMD 10, described in greater detail below.

IMD 10 may include one or more electrodes (not shown) on its housing, or may be coupled to one or more leads that carry one or more electrodes. System 2 may also include external devices 12. System 2 may be used to measure impedance and/or temperature to output a classification of a health condition of patient 4.

The example techniques may be used with IMD 10, which may be in wireless communication with at least one of external devices 12 or data servers 94. In some examples, IMD 10 is implanted outside of a thoracic cavity of patient 4 (e.g., subcutaneously in the pectoral location illustrated in FIG. 5). IMD 10 may be positioned near the sternum near or just below the level of the heart of patient 4, e.g., at least partially within the cardiac silhouette. IMD 10 may include a plurality of electrodes and may be configured for subcutaneous implantation outside of a thorax of patient 4.

Impedance measurements (e.g., taken via electrodes in the subcutaneous space) may be measurements of the impedance of interstitial fluid and subcutaneous tissue. In an example, during a heart failure decompensation event, reduction in cardiac output can tend to increase venous pressure. An increase in venous pressure tends to lead to an increase in pressure with respect to capillaries compared to the interstitial space. The combination of such tendencies may then lead to a net outflow of fluid from the capillaries into the interstitium or interstitial space of a patient. In such instances, the interstitium will have an increase in fluid accumulation. An increase in fluid accumulation tends to provide a reduction in impedance measured between electrodes.

IMD 10 may be configured to collect temperature measurements. For example, IMD 10 may include one or more of a thermocouple, a thermistor, a junction-based thermal sensor, a thermopile, a fiber optic detector, an acoustic temperature sensor, a quartz or other resonant temperature sensor, a thermo-mechanical temperature sensor, a thin film resistive element, etc. Changes in temperature may be indicative of, for example, an infection, a heart failure event, etc.

Implantable medical devices (IMDs) can sense and monitor impedance and temperature signals and use those signals to determine a health condition status of a patient, such as a heart condition, or other health condition status of a patient (e.g., edema, preeclampsia, hypertension, etc.). The sensors (e.g., electrodes, thermocouples, etc.) used by IMDs to sense impedance and temperature signals are typically integrated with a housing of the IMD and/or coupled to the IMD (e.g., via one or more leads). Example IMDs that include electrodes include the Reveal LINQ™ Insertable Cardiac Monitor (ICM), developed by Medtronic, Inc., of Minneapolis, MN, which may be inserted subcutaneously. Other example IMDs may include electrodes on a subcutaneous lead connected to another one of medical devices 17, such as a subcutaneous implantable cardioverter defibrillator (ICD) or an extravascular ICD. Such IMDs may facilitate relatively longer-term monitoring of patients during normal daily activities and may periodically transmit collected data to a network service, such as the Medtronic CareLink® Network.

Medical devices configured to measure impedance and temperature may implement the techniques of this disclosure for measuring impedance changes and temperature changes of a patient to determine whether the patient is experiencing worsening heart failure or decompensation. The techniques of this disclosure for identifying heart failure worsening may facilitate determinations of cardiac wellness and risk of sudden cardiac death and may lead to clinical interventions to suppress heart failure worsening, such as with medications.

IMD 10 may be configured to measure, in some cases among other physiological parameter values, impedance values within the interstitial fluid of patient 4. For example, IMD 10 may be configured to receive one or more signals indicative of subcutaneous tissue impedance from electrodes 16. In addition, IMD 10 may be configured to receive one or more signals indicative of temperature from temperature sensors, such as a thermocouple. In some examples, IMD 10 may be a purely diagnostic device. For example, IMD 10 may be a device that only determines subcutaneous impedance parameters and temperature parameters of patient 4, or a device that determines subcutaneous impedance parameters and temperature parameters as well as other physiological parameter values of patient 4. IMD 10 may use the impedance value measurements to determine one or more fluid index values, impedance scores, and/or various thresholds, such as adaptive thresholds, scoring thresholds, weighting factors for thresholds, and/or cardiac risk thresholds. IMD 10 may use the temperature measurements to determine core body temperature values, surface temperature values, external temperature values, etc.

Subcutaneous impedance may be measured by delivering a signal through an electrical path between electrodes. In some examples, the housing of IMD 10 may be used as an electrode in combination with electrodes located on leads. For example, system 2 may measure subcutaneous impedance by creating an electrical path between a lead and one of the electrodes. In additional examples, system 2 may include an additional lead or lead segment having one or more electrodes positioned subcutaneously or within the subcutaneous layer for measuring subcutaneous impedance. In some examples, two or more electrodes useable for measuring subcutaneous impedance may be formed on or integral with the housing of IMD 10.

System 2 may measure subcutaneous impedance of patient 4 and process impedance data to accumulate evidence of decreasing impedance. The accumulated evidence is referred to as a fluid index and may be determined as function of the difference between measured impedance values and reference impedance values. The fluid index may then be used to determine impedance scores that are indicative of a heart condition of patient 4. In an example, an impedance score may be measured against a risk threshold that identifies diagnostic states of the subcutaneous tissue impedance physiological parameters, which may be applied to probability model 19 as described herein. In some examples, subcutaneous impedance may provide information about fluid volume in the subcutaneous space, and in some instances, total blood volume, as well. In such examples, subcutaneous impedance measurements allow system 2 via probability model 19 to identify patients that have accumulated threshold levels of peripheral fluid as determined based on a plurality of evidence nodes, where at least one evidence node is based at least in part on a subcutaneous impedance measurement or subcutaneous impedance score.

In some examples, IMD 10 may also sense cardiac electrogram (EGM) signals via the plurality of electrodes and/or operate as a therapy delivery device. For example, IMD 10 may additionally operate as a therapy delivery device to deliver electrical signals to the heart of patient 4, such as an implantable pacemaker, a cardioverter, and/or defibrillator, a drug delivery device that delivers therapeutic substances to patient 4 via one or more catheters, or as a combination therapy device that delivers both electrical signals and therapeutic substances.

In some examples, system 2 may include any suitable number of leads coupled to IMD 10, and each of the leads may extend to any location within or proximate to a heart or in the chest of patient 4. For example, other examples therapy systems may include three transvenous leads and an additional lead located within or proximate to a left atrium of a heart. As other examples, a therapy system may include a single lead that extends from IMD 10 into a right atrium or right ventricle, or two leads that extend into a respective one of a right ventricle and a right atrium.

In some examples, IMD 10 may be implanted subcutaneously in patient 4. Furthermore, in some examples, external device 12 may monitor subcutaneous impedance values. In some examples, IMD 10 takes the form of the Reveal LINQ™ ICM, or another ICM similar to, e.g., a version or modification of, the LINQ™ ICM, which may be inserted subcutaneously. Such IMDs may facilitate relatively longer-term monitoring of patients during normal daily activities, and may periodically transmit collected data to a network service, such as the Medtronic CareLink® Network.

Although described in the context of examples in which IMD 10 includes an insertable or implantable IMD, example systems including one or more external devices of any type configured to sense subcutaneous tissue impedances may be configured to implement the techniques of this disclosure. In some examples, IMD 10 may be a device configured to measure impedances of a fluid and shifts in impedances of the fluid, such as interstitial fluid. For example, IMD 10 may have one or more electrodes disposed within one layer of patient 4 (e.g., subcutaneous layer), whereas at least one other electrode may be disposed within another layer of patient 4 (e.g., dermis layer, muscle layer, etc.). In such examples, IMD 10 may be able to measure impedances and shifts in impedances of the interstitial fluid of the subcutaneous layer. In another example, IMD 10 may be a cutaneous patch device having electrodes on the outside of the skin. In such examples, IMD 10 may use the cutaneous patch device to measure impedances and shifts in impedances of the interstitial fluid in the subcutaneous layer.

In examples in which IMD 10 also operates as a pacemaker, a cardioverter, and/or defibrillator, or otherwise monitors the electrical activity of the heart, IMD 10 may sense electrical signals attendant to the depolarization and repolarization of the heart of patient 4 via electrodes of or coupled to IMD 10, e.g., which may include the electrodes used to determine subcutaneous impedance. In some examples, IMD 10 can provide pacing pulses to the heart of patient 4 based on the electrical signals sensed within the heart of patient 4. The configurations of electrodes used by IMD 10 for sensing and pacing may be unipolar or bipolar. IMD 10 may also provide defibrillation therapy and/or cardioversion therapy via electrodes located on at least one lead, as well as a housing electrode. IMD 10 may detect tachyarrhythmia of the heart of patient 4, such as fibrillation of atria or ventricles, and deliver defibrillation or other tachyarrhythmia therapy to the heart of patient 4 in the form of electrical pulses. In some examples, IMD 10 may be programmed to deliver a progression of therapies, e.g., pulses with increasing energy levels, until a fibrillation of the heart of patient 4 is stopped. IMD 10 detects fibrillation or other tachyarrhythmias employing tachyarrhythmia detection techniques known in the art.

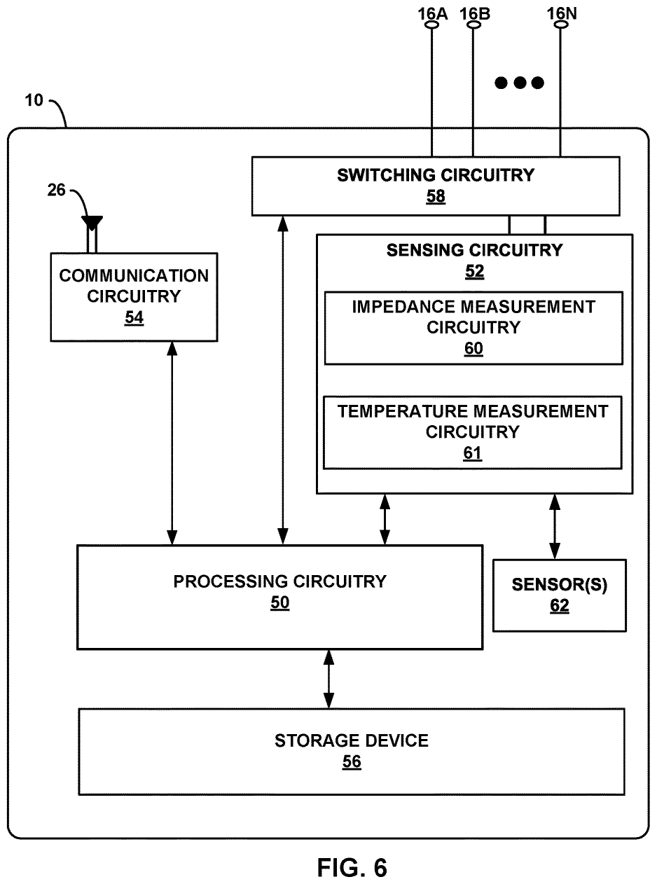

FIG. 6 is a functional block diagram illustrating an example configuration of IMD 10. IMD 10 may include an example of one of medical devices 17. In the illustrated example, IMD 10 includes electrodes 16A-16N (collectively, “electrodes 16”), antenna 26, processing circuitry 50, sensing circuitry 52, impedance measurement circuitry 60, communication circuitry 54, storage device 56, switching circuitry 58, sensors 62. IMD 10, along with other medical devices 17, may also include a power source. In general, the power source may include a rechargeable or non-rechargeable battery. Each of medical devices 17 may include components common to those of IMD 10. For example, each of medical devices 17 may include processing circuitry 50. For sake of brevity, each configuration of each medical devices 17 will not be described in this application. That is, certain components of IMD 10 may serve as representative components of other medical devices 17 (e.g., storage device 56, communication circuitry 54, sensors 62, etc.).

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16 via switching circuitry 58, e.g., to select the electrodes 16 and polarity, referred to as the sensing vector, used to sense impedance and/or cardiac signals, as controlled by processing circuitry 50. Sensing circuitry 52 may sense signals from electrodes 16, e.g., to produce a cardiac EGM, in order to facilitate monitoring the electrical activity of the heart. Sensing circuitry 52 also may monitor signals from sensors 62, which may include one or more accelerometers, pressure sensors, and/or optical sensors, as examples. Sensors 62 may further include one or more temperature sensing devices. Any suitable sensors 62 may be used to detect temperature or changes in temperature. In some examples, sensors 62 may include a thermocouple, a thermistor, a junction-based thermal sensor, a thermopile, a fiber optic detector, an acoustic temperature sensor, a quartz or other resonant temperature sensor, a thermo-mechanical temperature sensor, a thin film resistive element, etc.

In some examples, sensing circuitry 52 may include one or more filters and amplifiers for filtering and amplifying signals received from sensors 62 and/or electrodes 16. For example, sensing circuitry 52 may include one or more low-pass filters having various cutoff frequencies predefined to apply to temperature values obtained from sensors 62, such as from one or more temperature sensors. In some examples, sensing circuitry 52 may include circuitry configured to digitally filter measured temperature values using one or more cutoff frequencies, or otherwise using one or more different filtering processes to achieve different degrees of smoothing of a series of temperature values. For example, sensing circuitry 52 may include certain processing circuitry, such as processing circuitry 50, configured to smooth temperature values determined over time to create smoothened temperature signals. In some examples, processing circuitry of sensing circuitry 52 may perform smoothing of temperature values measured by sensors 62, such that processing circuitry 50 may perform various other techniques of this disclosure based on the smoothened temperature signals. In some examples, processing circuitry 50 may include sensing circuitry 52 with processing circuitry 50 being configured to smooth temperature values determined over time to create smoothened temperature signals (e.g., by performing digital and/or analog filtering).

Processing circuitry 50 may cause sensing circuitry 52 to periodically measure a physiological parameters or other parameter values of IMD 10, such as impedance values and temperature values. Processing circuitry 50 may control sensing circuitry 52 to obtain impedance and temperature measurements via one or more of electrodes 16 or sensors 62.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network.

Antenna 26 and communication circuitry 54 may be configured to transmit and/or receive signals via inductive coupling, electromagnetic coupling, NFC technologies, RF communication, Bluetooth®, Wi-Fi™, or other proprietary or non proprietary wireless communication schemes. In some examples, processing circuitry 50 may provide data to be uplinked to external device 12 via communication circuitry 54 and control signals using an address/data bus. In another example, communication circuitry 54 may provide received data to processing circuitry 50 via a multiplexer.

In some examples, processing circuitry 50 may send impedance data and/or temperature data to external devices 12 or data servers 94 via communication circuitry 54. For example, IMD 10 may send external devices 12 or data servers 94 collected impedance measurements. External devices 12 and/or data servers 94 may then analyze those impedance measurements. In some examples, processing circuitry 50 may receive temperature values of patient 4 from one or more other devices via communication circuitry 54. In some examples, the one or more other devices may include a sensor device, such as an activity sensor, heart rate sensor, a wearable device worn by patient 4, an external temperature sensor (e.g., a digital thermometer configured to communicate with processing circuitry 50), etc. That is, the one or more other devices may, in some examples, be external to IMD 10.

In other example, processing circuitry 50 may send temperature data to external device 12 via communication circuitry 54. For example, IMD 10 may send external device 12 collected temperature measurements, which are then analyzed by external device 12. In such examples, external device 12 performs the processing techniques described herein. Alternatively, IMD 10 may perform the processing techniques and transmit the processed temperature data and/or classifications of whether heart failure is detected to external device 12 for reporting purposes, e.g., for providing an alert to patient 4 or another user.

In some examples, processing circuitry 50 may use switching circuitry 58 to select, e.g., via a data/address bus, which of the available electrodes are to be used to obtain impedance measurements of interstitial fluid and to sense cardiac signals, and to select the polarities of the electrodes. Switching circuitry 58 may include a switch array, switch matrix, multiplexer, transistor array, microelectromechanical switches, or any other type of switching device suitable to selectively couple sensing circuitry 58 to selected electrodes. In some examples, sensing circuitry 52 includes one or more sensing channels, each of which may include an amplifier. In response to the signals from processing circuitry 50, switching circuitry 58 may couple the outputs from the selected electrodes to one of the sensing channels.

Sensing circuitry 52 may include impedance measurement circuitry 60. Processing circuitry 50 may control impedance circuitry 60 to periodically measure an electrical parameter to determine an impedance, such as a subcutaneous impedance indicative of fluid found in an interstitium. For a subcutaneous impedance measurement, processing circuitry 50 may control impedance measurement circuitry 60 to deliver an electrical signal between selected electrodes 16 and measure a current or voltage amplitude of the signal. Processing circuitry 50 may select any combination of electrodes 16, e.g., by using switching circuitry 58 and sensing circuitry 52. Impedance measurement circuitry 60 includes sample and hold circuitry or other suitable circuitry for measuring resulting current and/or voltage amplitudes. Processing circuitry 50 determines an impedance value from the amplitude values received from impedance measurement circuitry 60.

As used herein, the term “impedance” is used in a broad sense to indicate any collected, measured, and/or calculated value that may include one or both of resistive and reactive components. In some examples, subcutaneous tissue impedance parameters are derived from subcutaneous tissue impedance signals received from electrodes 16.

Sensing circuitry 52 may include temperature measurement circuitry 61. Processing circuitry 50 may control temperature measurement circuitry 61 to measure values on a periodic basis, such as on an hourly basis, daily basis, weekly basis, or the like. In one example, sensing circuitry 52 may measure temperature values during a particular portion of a day. As an example, sensing circuitry 52 may control temperature measurement circuitry 61 to measure temperature values every twenty minutes for a predetermined number of hours, such as between noon and 5 pm. Processing circuitry 50 may determine a final measured temperature value by calculating an average of the measurements. In this case, the daily value may be the average of the temperature values measured by sensing circuitry 52 during the day (e.g., within a 24-hr time period, within a 24-hr time period where measurements are selectively taken between particular times and/or in response to certain triggers, etc.).

In some examples, sensing circuitry 52 may be configured to sample temperature measurements at a particular sampling rate. In such examples, sensing circuitry 52 may be configured to perform downsampling of the received temperature measurements. For example, sensing circuitry 52 may perform downsampling in order to decrease the throughput rate for processing circuitry 50. This may be particularly advantageous where sensing circuitry 52 has a high sampling rate when active.

As used herein, the term “temperature value” is used in a broad sense to indicate any collected, measured, and/or calculated value. In some examples, temperature values are derived from temperature signals received from one or more of sensors 62. For example, temperature values may include an average (e.g., mean, mode, standard deviation) of temperature signals received from one or more of sensors 62.

Processing circuitry 50 may also determine other physiological parameter values that can be used to determine probability of a health event based on the cardiac EGM and detection of depolarizations therein. As examples, processing circuitry 50 may determine one or more heart rate values, such as night heart rate values, one or more heart rate variability values. As other examples, processing circuitry 50 may determine magnitudes of or intervals between features within the cardiac EGM, such as depolarization amplitudes, depolarization widths, or intervals between depolarizations and repolarizations.

In some examples, sensors 62 include one or more accelerometers or other sensors configured to generate signals that indicate motion and orientation of patient 4, e.g., that indicate activity level or posture of the patient. In some examples, processing circuitry 50 processes such signals to determine values of one or more physiological parameters that can be used to determine probability of a health event. For example, processing circuitry 50 may quantify duration, frequency, and/or intensity of activity and/or posture changes, e.g., daily or during some other period. In some examples, processing circuitry 50 may determine an amount of time patient spends inactive, e.g., sleeping, but not in a supine posture based on such signals.

Because either IMD 10 or external device 12 may be configured to include sensing circuitry 52, impedance measurement circuitry 60 and temperature measurement circuitry 61 may be implemented in one or more processors, such as processing circuitry 50 of IMD 10 or processing circuitry of external devices 12. Impedance measurement circuitry 60 and temperature measurement circuitry 61, in this example, shown in conjunction with sensing circuitry 52 of IMD 10. Impedance measurement circuitry 60 and temperature measurement circuitry 61 may be embodied as one or more hardware modules, software modules, firmware modules, or any combination thereof.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media. For example, storage device 56 may include random access memory (RAM), read-only memory (ROM), non-volatile RAM (NVRAM), electrically-erasable programmable ROM (EEPROM), erasable programmable ROM (EPROM), flash memory, or any other digital media. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include impedance values and/or digitized cardiac EGMs, as examples.

In some examples, processing circuitry, e.g., processing circuitry 50 of IMD 10, processing circuitry 80 of external device 12, or processing circuitry 98 of data servers 94, may determine RRs or other respiration parameters based on analysis of impedance values determined as described herein but, in some cases, sampled at a higher rate than for detecting changes in the fluid status of patient 4. For example, processing circuitry 50 (or processing circuitry of another device) may employ any of a variety of techniques to detect the frequency, period between, or magnitude of fluctuations in the impedance values associated with respiration of patient 4. In some examples, processing circuitry 50 may control impedance measurements for determining respiration parameters to occur when certain conditions are satisfied, e.g., time of day, such as night, or patient activity level or posture.

In some examples, processing circuitry, e.g., processing circuitry 50 of IMD 10, processing circuitry 80 of external device 12, or processing circuitry 98 of data servers 94, may determine a diagnostic state based on the impedance parameters and the temperature parameters In some examples, processing circuitry 98 may determine the diagnostic state periodically, such as at multiple intervals each day. In yet another example, processing circuitry 98 may determine the diagnostic state at longer intervals, such as once a week or once every two weeks.

In some examples, processing circuitry 98 may determine a heart failure risk status. The risk status may be determined as low, medium, high, etc. In some examples, processing circuitry 98 may use a different number of risk categories, such as including a category for very high risk in some instances or very low risk. In addition, processing circuitry 98 may not include certain categories, such as the medium risk category, and instead only monitor low and high-risk categories. In a non-limiting example, processing circuitry 98 may determine risk status as follows: low risk if the diagnostic state for impedance is dry and the diagnostic state for temperature is hot; medium risk if the diagnostic state for impedance is dry and the diagnostic state for temperature is cold; medium risk if the diagnostic state for impedance is wet and the diagnostic state for temperature is hot; and high risk if the diagnostic state for impedance is wet and the diagnostic state for temperature is cold.

In some examples, processing circuitry, e.g., processing circuitry 50 of IMD 10, processing circuitry 80 of external device 12, or processing circuitry 98 of data servers 94, may determine satisfaction of at least one of: a scoring threshold and an impedance threshold, with respect to one or more time windows. For example, processing circuitry 98 may modify an impedance score in response to one or more fluid index values satisfying one or more scoring thresholds for at least one of: a predetermined amount of time and a predetermined number of times (e.g., number of days, etc.). In an illustrative example, processing circuitry 98 may increment the impedance score by a point value (e.g., a 1 point value) in response to the following example conditions (e.g., scoring thresholds) being satisfied with respect to the first time period: (1) the fluid index values in the last 30 days have been greater than or equal to the adaptive threshold (multiplied by 0.6) for one or more days; (2) the fluid index values in the last 30 days have been greater than or equal to the adaptive threshold (multiplied by 1.7) for one or more days; or (3) the fluid index values in the last 30 days have been greater than or equal to the adaptive threshold (multiplied by 3.2) for one or more days. In this example, processing circuitry 98 determined the weighting factors as 0.6, 1.7, and 3.2. In this example, the first time period is the last 30 days. However, as discussed herein, the time periods and the weighting factors may vary depending on specifics related to patient 4, for example.

In another example, processing circuitry 98 may also increment the impedance score by a point value of greater than one (e.g., two points) in response to the average impedance satisfying an impedance value threshold and the fluid index satisfying scoring thresholds. In some examples, processing circuitry 98 may modify the impedance score in response to the average impedance value satisfying an impedance value threshold. The impedance value threshold may, in some examples, be less than or equal to approximately 600 ohms or another comparable ohm value.

For example, processing circuitry 98 may increment the impedance score by two points in response to the following example conditions (e.g., scoring thresholds and impedance value thresholds) being met with respect to the first time period: (1) the fluid index values in the last 30 days have been greater than or equal to the adaptive threshold (multiplied by 1.5) for 24 or more days; or (2) the average impedance in the last 30 days has been less than or equal to approximately 600 ohms. For the first condition, the 24 or more days may be consecutive days or instead may be a cumulative 24 days. For the average impedance, the average impedance in the last 30 days may refer to a set of daily average impedances in the last 30 days. In some examples, the average impedance in the last 30 days may refer to a single average of the impedance values measured over time. In another example, the average impedance may refer to a single average of the daily average impedance values determined over time.

In some examples, processing circuitry 98 may determine anew or modify an impedance score when the fluid index values during the second time period satisfy the adaptive threshold multiplied by the corresponding weighting factors. In addition, processing circuitry 98 may determine anew or modify an impedance score when the average impedance satisfies an impedance threshold during the second time period.

In an illustrative example, processing circuitry 98 may increment the impedance score by a point value equal to one in response to the following example conditions being satisfied with respect to the second time period: (1) the fluid index values in the last seven days have been greater than or equal to the adaptive threshold (multiplied by 0.6) for one or more days; (2) the fluid index values in the last seven days have been greater than or equal to the adaptive threshold (multiplied by 1.7) for one or more days; or (3) the fluid index values in the last seven days have been greater than or equal to the adaptive threshold (multiplied by 1.5) for seven or more days. In this example, IMD 10 determined the weighting factors as 0.6, 1.7, and 1.5. In this example, the second time period is the last seven days. However, as discussed herein, the time periods and the weighting factors may vary depending on specifics related to patient 4, for example. In addition, for the last condition, the 7 or more days may be consecutive days or instead may be a cumulative 7 days.

In another example, IMD 10 may also increment the impedance score by a point value of greater than one (e.g., two points) in response to other example conditions being met with respect to the second time period: (1) the fluid index values in the last seven days have been greater than or equal to the adaptive threshold (multiplied by 3.2) for one or more days; or (2) the average impedance in the last seven days has been less than or equal to approximately 600 ohms. For the average impedance, the average impedance in the last 7 days may refer to a set of daily average impedances in the last 7 days. In some examples, the average impedance in the last 7 days may refer to a single average of the impedance values measured over time. In another example, the average impedance may refer to a single average of the daily average impedance values determined over time.

In some examples, where overlaps exist between conditions, only the higher point value would be added to the impedance score so as to avoid any compounding affects to the modification of the impedance score. In keeping with the example described above, where two conditions are met (e.g., average impedance in last 7 days and in the last 30 days has been greater than or equal to approximately 600 ohms), the impedance score may only increment by two and not by four. In other examples, where two conditions are met (e.g., average impedance in last 7 days and in the last 30 days has been greater than or equal to approximately 600 ohms), IMD 10 may increment the impedance score based on both conditions being satisfied. The impedance score may be then be used to determine a diagnostic state of the subcutaneous tissue impedance physiological parameter to serve as one of evidence nodes 8.

The above techniques of determining an impedance score may also be performed on a periodic basis. For example, the impedance scores may be determined according to a resolution parameter setting of processing circuitry 50 (e.g., the resolution parameter used to signal a frequency at which electrodes 16 should probe for impedance measurements). In other examples, the impedance score may be calculated irrespective of the resolution parameter, which, for example, may apply to the fluid index determination and/or the reference impedance value determination, but not the impedance score determination. For instance, processing circuitry 50 may calculate the impedance scores at several time intervals each day (e.g., once in the morning, once in the afternoon, once in the evening, once after meals, etc.). In some examples, processing circuitry 50 may calculate the impedance score once a day, each week, every two weeks, each month, etc. In some examples, processing circuitry 50 may also calculate the impedance score in response to a user command (e.g., from a physician, from a user interface) or in response to a satisfaction of another condition (e.g., based on activity level or other physiological parameters). For example, processing circuitry 50 may determine impedance score on a per measurement basis, such as on a per fluid index determination basis or on a per impedance measurement basis. A person of skill in the art should appreciate that various periods may exist for when IMD 10, data servers 94, or external device 12 may transmit impedance scores, receive impedance scores, receive fluid index values, and/or otherwise, calculate impedance scores for subsequent analysis.

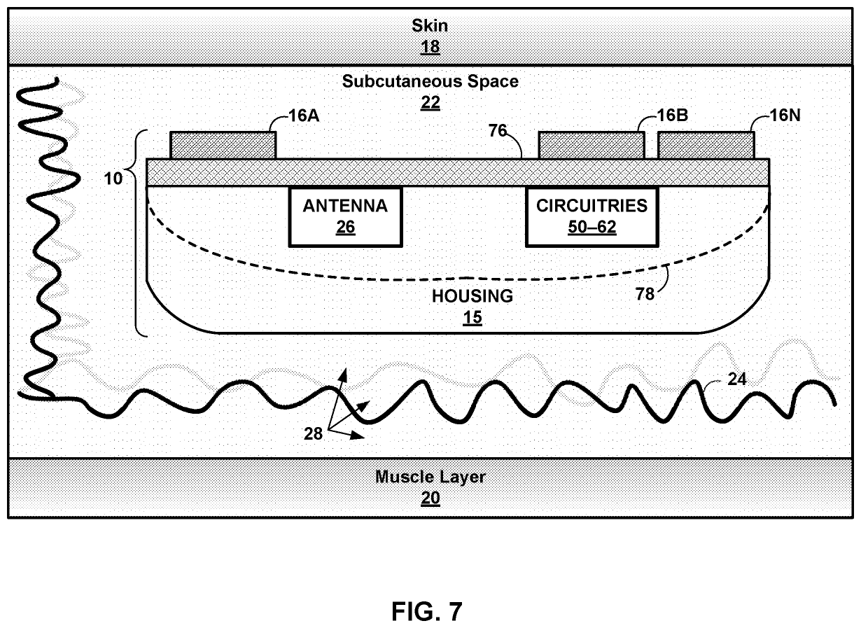

FIG. 7 is a conceptual side view diagram illustrating an example configuration of IMD 10. The conceptual side view diagram illustrates a muscle layer 20 and a skin layer 18 (e.g., dermis layer, epidermis layer). The region between muscle layer 20 and skin layer 18 includes a subcutaneous space 22. Subcutaneous space includes blood vessels 24, such as capillaries, arteries, or veins, and interstitial fluid in an interstitium 28 of subcutaneous space 22. Subcutaneous space 22 has interstitial fluid that is commonly found between skin 18 and muscle layer 20. Subcutaneous space 22 may include interstitial fluid that surrounds blood vessels 24. For example, interstitial fluid surrounds capillaries and allows the passing of capillary elements (e.g., nutrients) between the different layers of a body through interstitium 28.

In the example shown in FIG. 7 IMD 10 may include a leadless, subcutaneously implantable monitoring device having a housing 15 and an insulative cover 76. Electrodes 16 may be formed or placed on an outer surface of cover 76. Although the illustrated example includes three electrodes 16, IMDs including or coupled to more or less than three electrodes 16 may implement the techniques of this disclosure in some examples. In some examples, electrodes 16 may be disposed all within a single layer, such as subcutaneous space 22 and contact interstitial fluid in subcutaneous space 22.

Circuitries 50-62 may be formed or placed on an inner surface of cover 76, or within housing 15. In the illustrated example, antenna 26 is formed or placed on the inner surface of cover 76, but may be formed or placed on the outer surface in some examples. In some examples, one or more of sensors 62 may be formed or placed on the outer surface of cover 76. In some examples, insulative cover 76 may be positioned over an open housing 15 such that housing 15 and cover 76 enclose antenna 26 and circuitries 50-62, and protect antenna 26 and circuitries from fluids such as interstitial fluids or other bodily fluids.

Sensors 62 may include one or more temperature sensing devices fixed to an outer housing 15 of IMD 10 or insulative cover 76, instead of or in addition to temperatures sensors within housing 15. In a non-limiting example, IMD 10 may include more than two temperature sensing devices on the inside of IMD 10 and in addition, may include more than two temperature sensing device on the outside of IMD 10. In some examples, temperature data obtained from multiple temperature sensing device may be averaged or otherwise combined to obtain a representative temperature signal that may also be smoothed as discussed elsewhere in this disclosure.

One or more of antenna 26 or circuitries 50-62 may be formed on the inner side of insulative cover 76, such as by using flip chip technology. Insulative cover 76 may be flipped onto a housing 15. When flipped and placed onto housing 15, the components of IMD 10 formed on the inner side of insulative cover 76 may be positioned in a gap 78 defined by housing 15. Electrodes 16 may be electrically connected to switching circuitry 58 through one or more vias (not shown) formed through insulative cover 76. Insulative cover 76 may be formed of sapphire (i.e., corundum), glass, parylene, and/or any other suitable insulating material. Housing 15 may be formed from titanium or any other suitable material (e.g., a biocompatible material). Electrodes 16 may be formed from any of stainless steel, titanium, platinum, iridium, or alloys thereof. In addition, electrodes 16 may be coated with a material such as titanium nitride or fractal titanium nitride, although other suitable materials and coatings for such electrodes may be used.

In a non-limiting example, one or more temperature sensing devices may be formed or placed on the outer surface of housing 15 or insulative cover 76, with additional sensors 62, such as one or more additional temperature sensing devices, may be formed within housing 15, such as on a printed circuit board (PCB) disposed within housing 15. In some examples, a temperature sensing device may be formed or placed on the outer surface of housing 15 or insulative cover 76 using a connection interface. The connection interface, in some instances, may include a wired connection interface. For example, a temperature sensing device may be placed on the outer surface of housing 15 or insulative cover 76 using a press fit connector, solder paste, conductive mounting pins, input-output cables or other wire connectors, threaded connectors, wire pads, press-in pins, etc., or various combinations thereof. In an example involving a wireless connection interface, the temperature sensing device may include communication and processing circuitry to transmit temperate values to one or more other devices, such as to communication circuitry 54, communication circuitry 82 or otherwise over network 92. In one illustrative example, sensors 62 on an outer surface of cover 76 may be connected to circuitry within housing 15 through one or more vias (not shown) formed through insulative cover 76.

One or more temperature sensing devices may be on or in the patient. In examples where multiple temperature sensing devices are on the same device, the temperature sensors may be oriented superficially and internally. In examples where the multiple temperature sensing devices are on separate implanted devices, the respective temperature sensors of the devices implanted at different depths may indicate changes in peripheral perfusion (e.g., vasoconstriction, shock, etc.). Bilateral implants at same depth may identify pocket infection. In examples where the multiple temperature sensing devices are on separate devices, at least one of the devices being a wearable device and at least one of the devices being an implanted device, temperature on extremities compared to subcutaneous temperature may indicate change in perfusion. The temperature data from these multiple sources may also identify and filter out environmental changes. Example systems and techniques for using multiple temperature sensors are described in commonly-assigned U.S. patent application Ser. No. 16/751,929, filed on Jan. 24, 2020 and titled “IMPLANTABLE MEDICAL DEVICE USING TEMPERATURE SENSOR TO DETERMINE INFECTION STATUS OF PATIENT,” the entire content of which is incorporated herein by reference. Additionally, example techniques for detecting and mitigating changes in the pose (e.g., position and/or orientation) of sensors, such as temperature and impedance sensors, within a patient are described in commonly-assigned U.S. patent application Ser. No. 17/101,945, filed on Nov. 23, 2020 and titled “DETECTION AND MITIGATION OF INACCURATE SENSING BY AN IMPLANTED SENSOR OF A MEDICAL SYSTEM,” the entire content of which is incorporated herein by reference.

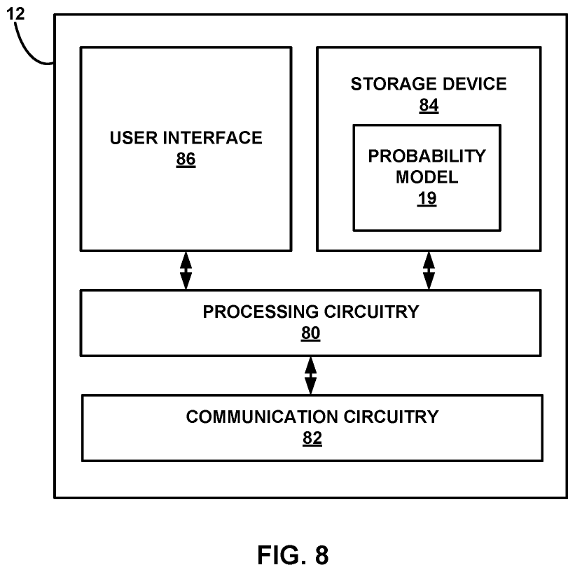

FIG. 8 is a block diagram illustrating an example configuration of components of external device 12. In some examples, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as one of medical devices 17 (e.g., IMD 10). Under the control of processing circuitry 80, communication circuitry 82 may receive downlink telemetry from, as well as send uplink telemetry to, one of medical devices 17 (e.g., IMD 10), or another device (e.g., data servers 94). Communication circuitry 82 may be configured to transmit or receive signals via inductive coupling, electromagnetic coupling, near field communication (NFC) technologies (e.g., inductive coupling, NFC or other communication technologies operable at ranges less than 10-20 cm), RF communication, Bluetooth®, Wi Fi™, or other proprietary or non-proprietary wireless communication schemes. Communication circuitry 82 may also be configured to communicate with devices other than medical devices 17 via any of a variety of forms of wired and/or wireless communication and/or network protocols.

Storage device 84 may be configured to store information within external device 12 during operation. Storage device 84 may include a computer readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external devices 12 to temporarily store information during program execution.

Storage device 84 may store one or more probability models 19. Additionally or alternatively, other storage devices described herein (e.g., storage device 56, storage device 96, etc.) may store probability models 19. Storage device 84 may also store historical data, diagnostic state data, physiological parameter values, probability scores, etc.

Data exchanged between external devices 12 and medical devices 17 may include operational parameters (e.g., physiological parameter values, diagnostic states, etc.). External devices 12 may transmit data including computer readable instructions which, when implemented by medical devices 17, may control medical devices 17 to change one or more operational parameters and/or export collected data (e.g., physiological parameter values). For example, processing circuitry 80 may transmit an instruction to medical devices 17 which requests medical devices 17 to export collected data (e.g., impedance data, fluid index values, and/or impedance scores, temperature values, blood pressure, ECG records, etc.) to external devices 12.

In turn, external devices 12 may receive the collected data from medical devices 17 and store the collected data in storage device 84. Processing circuitry 80 may implement any of the techniques described herein to model physiological parameter values received from medical devices 17 to determine diagnostic states, probability scores, etc. Using the modeling techniques disclosed herein, processing circuitry 80 may determine a likelihood that the patient is experiencing an adverse health event (e.g., heart failure decompensation) or is likely to experience an adverse health event within a predetermined amount of time (e.g., within the next 3 days, 7 days, 10 days, 30 days, 40 days, etc.). In an illustrative example, the predetermined amount of time may be at least approximately 7 days from when the probability score is determined, such that the probability score indicates the likelihood that an adverse health event will occur in the next 7 days or indicates that the patient is likely already experiencing an adverse health event, such as heart failure decompensation.

External devices 12 may be a computing device with a display viewable by a user and an interface for providing input to external devices 12 (i.e., a user input mechanism). The user may be a physician technician, surgeon, electrophysiologist, clinician, or patient 4. In some examples, external devices 12 may be a notebook computer, tablet computer, computer workstation, one or more servers, cellular or “smart” phone, smartwatch, personal digital assistant, handheld computing device, networked computing device, or another computing device that may run an application that enables the computing device to interact with IMD 10. External devices 12 may be configured to communicate with IMD 10 and, optionally, another computing device, via wired or wireless communication. External devices 12, for example, may communicate via NFC technologies (e.g., inductive coupling, NFC or other communication technologies operable at ranges less than 10-20 cm) and far-field communication technologies (e.g., Radio Frequency (RF) telemetry according to the 802.11 or Bluetooth® specification sets, or other communication technologies operable at ranges greater than NFC technologies). In some examples, external devices 12 may include a programming head that may be placed proximate to the body of patient 4 near the IMD 10 implant site in order to improve the quality or security of communication between IMD 10 and external device 12.

In one example, a user, such as a clinician or patient 4, may interact with external devices 12 through user interface 86. User interface 86 includes a display (not shown), such as a liquid crystal display (LCD) or a light emitting diode (LED) display or other type of screen, with which processing circuitry 80 may present information related to medical devices 17 (e.g., cardiac EGMs, blood pressure, subcutaneous impedance values, RR, etc.). In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external devices 12 and provide input. In other examples, user interface 86 also includes audio circuitry for providing audible notifications, instructions or other sounds to the user, receiving voice commands from the user, or both.

In some examples, user interface 86 of external devices 12 may receive input from the user. The user interface may include, for example, a keypad and a display, which may for example, be a cathode ray tube (CRT) display, an LCD, or an LED display. The keypad may take the form of an alphanumeric keypad or a reduced set of keys associated with particular functions. External devices 12 can additionally or alternatively include a peripheral pointing device, such as a mouse, via which the user may interact with the user interface. In some examples, a display of external devices 12 may include a touch screen display, and a user may interact with external devices 12 via the display. It should be noted that the user may also interact with external devices 12 remotely via a networked computing device.

In some examples, external devices 12 may be coupled to external electrodes, or to implanted electrodes via percutaneous leads. In some examples, external devices 12 may monitor subcutaneous tissue impedance measurements from IMD 10. External devices 12 may be used to configure operational parameters for IMD 10. For example, external device 12 may provide a parameter resolution for IMD 10 that indicates a resolution of data that IMD 10 should be obtaining. Examples of resolution parameters may include a frequency at which the electrodes process impedance measurements or a frequency at which impedance measurements should be considered in determining a diagnostic state.

In some examples, external devices 12 may include one or more of a thermocouple, a thermistor, or another type of thermal sensor. In some examples, external devices 12 may monitor temperature measurements from IMD 10. External devices 12 may be configured to obtain external temperature measurements from other devices, such as other external computing devices, in order to calibrate temperature measurements from IMD 10.

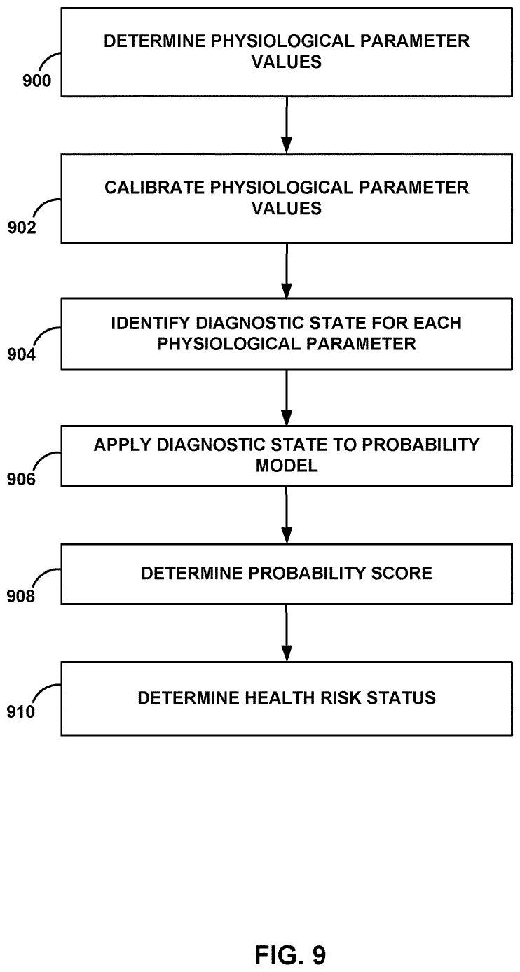

FIG. 9 illustrates an example method that may be performed by one or more of medical devices 17, external device 12, and/or data servers 94 in conjunction with probability model 19 to determine a probability score with respect to patient 4, in accordance with one or more techniques disclosed herein. Although described as being performed by data servers 94, one or more of the various example techniques described with reference to FIG. 10 may be performed by any one or more of medical devices 17, external device 12, or data servers 94, e.g., by the processing circuitry of any one or more of these devices.

In some examples, processing circuitry, e.g., processing circuitry 50 of IMD 10, processing circuitry 80 of external device 12, or processing circuitry 98 of data servers 94, may determine values of physiological parameters, such as those physiological parameters described herein (900). Processing circuitry 98 may calibrate the physiological parameters (902). For example, processing circuitry 98 may apply feature extraction model 27, which may perform principal component analysis to determine and remove the influence of environmental factors that are affecting measurements from medical devices 17. For each of the physiological parameters, processing circuitry 98 may identify diagnostic states 11 (904). In some instances, processing circuitry 98 may filter irrelevant physiological parameters or parameter values prior to identifying diagnostic states or after identifying diagnostic states. For example, certain physiological parameters or values thereof may not be relevant to the purpose of deploying probability model 19. In such instances, those physiological parameters or values may still be available in storage device 96, but processing circuitry 98 may determine those parameters should not be used as evidence nodes 8. Processing circuitry 98 may apply the diagnostic states to probability model 19 (906). For example, processing circuitry 98 may access probability model 19 stored in storage device 96 (or in another storage device, such as a storage device of medical devices 17, external devices 12, a cloud computing system, etc.) and execute probability model 19. Processing circuitry 98 may execute probability model 19 in response to a user command or may do so automatically in response to a triggering event. For example, processing circuitry 98 may determine that all necessary diagnostic states have been determined and that the probability model 19 is ready for execution. In some examples, processing circuitry 98 may receive data for various physiological parameters or processing circuitry 98 may access data from storage device 96. For the particular set of physiological parameters used (e.g., parameters having sufficient data), processing circuitry 98 may use the diagnostic states to determine probability score 25. In some examples, processing circuitry 98 may use probability score 25 to train probability model 19 for future rounds of determining probability scores. For example, processing circuitry 98 may use the current or incoming data to determine prior probability values 21.

In some examples, processing circuitry 98 may execute probability model a number of times in a finite period of time in order to determine an average probability score for the finite period of time. In some instances, processing circuitry 98 may execute probability model in accordance with a Monte Carlo simulation (e.g., using repeated random sampling to obtain numerical results).

As such, processing circuitry 98 may determine one or more probability scores from probability model 19 (908). Processing circuitry 98 may use evidence nodes 8 as input to probability model 19 to determine a probability score that indicates a likelihood that a patient will experience an adverse health event within a predetermine period of time (e.g., within 30 days of determining the probability score). As discussed herein, the probability model may use the prior probability value and a conditional likelihood parameter as additional inputs to determine the probability score. The probability score (p value) may be represented as a decimal value. In some examples, processing circuitry may then update the probability model using the determined probability score. In some examples, a Bayesian ML model may determine the probability score, where the ML model may be trained on prior probability values and feedback received regarding the accuracy of the probability score in predicting adverse health events.

Processing circuitry 98 may compare the probability score to at least one risk threshold. Based on the comparison, processing circuitry 98 may determine one of plurality of discrete risk categorizations. For example, discrete risk categorizations may be high risk, low risk, or medium risk. In this way, processing circuitry 98 may determine a health risk status for a patient based at least in part on the probability score (910). In an example, processing circuitry 98 may be configured to monitor a patient, for example, with heart failure for a developing heart failure decompensation in accordance with one or more of the various techniques disclosed herein to then allow for early intervention potentially before clinical symptoms are experienced or hospitalization is needed. In another example, through implementation of the disclosed probability model (e.g., a Bayesian model), processing circuitry 98 may combine information from multiple “orthogonal” parameters, that alone may not be very specific, to ultimately gain a more specific classification.

It should be noted that one or more of the various example techniques described with reference to FIG. 9 may be performed on a periodic basis. For example, the probability score may be determined according to a resolution parameter setting of medical devices 17 and/or for patient 4. In other examples, the probability score may be calculated irrespective of the resolution parameter. Data servers 94 may calculate the probability score once a day, each week, every two weeks, each month, etc. In some examples, data servers 94 may also calculate the probability score in response to a user command (e.g., from a physician, from a user interface) or in response to a satisfaction of another condition, such as upon receiving or determining a particular number of diagnostic states, or an indication of a change in the condition of patient from a source other than the application of the probability model.

Data servers 94 may also trigger a probability score determination when an activity level or other physiological parameter of patient 4 satisfies a threshold (e.g., low activity when patient 4 is resting or sleeping). In one other example, data servers 94 or medical device 17, e.g., IMD 10, may determine the probability score of patient 4 on a per measurement basis, such as on a per impedance score determination basis. A person of skill in the art would appreciate that various periods may exist for when IMD 10 or external device 12 may determine a probability score in accordance with FIG. 9, transmit a probability score, etc. In some examples, data servers 94 may determine the probability score of patient 4 in response to receipt of physiological parameter data for patient via network 92, e.g., from medical devices 17.

In addition, although described in terms of data servers 94 performing one or more of the various example techniques of this disclosure, it is to be understood that any number of different components described may perform one or more of the various example techniques of this disclosure. For example, IMD 10 or external device 12 may determine diagnostic states 11 or physiological parameter values. In addition, data servers 94 may include multiple computing devices (e.g., a remote cloud server) that collectively determines probability scores of patient 4 experiencing an adverse health event within a predetermined period of time.

Various examples have been described. However, one of ordinary skill in the art will appreciate that various modifications may be made to the described examples without departing from the scope of the claims. For example, additional physiological parameters may be considered to determine probability scores of worsening heart failure or other adverse health events. Examples of other physiological parameters are described in U.S. application Ser. Nos. 12/184,149 and 12/184,003 by Sarkar et al., entitled “USING MULTIPLE DIAGNOSTIC PARAMETERS FOR PREDICTING HEART FAILURE EVENTS,” and “DETECTING WORSENING HEART FAILURE BASED ON IMPEDANCE MEASUREMENTS,” both filed on Jul. 31, 2008, both of which are incorporated herein by reference in their entirety.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

The following examples are a non-limiting list of clauses in accordance with one or more techniques of this disclosure.

Example 1. A medical system comprising: an implantable medical device; an external device; a data server; and processing circuitry configured to: determine a respective one or more values for each of a plurality of physiological parameters, the plurality of physiological parameters comprising: fluid retention, wherein the respective one or more values for fluid retention correspond to a fluid retention state from a first set of states comprising a plurality of fluid retention states; and temperature, wherein the respective one or more values for temperature correspond to a temperature state from a second set comprising a plurality of temperature states; determine, using a probability model, one or more diagnostic states based on the respective values; determine a classification of a health condition of a patient based on the application of the diagnostic states to the probability model; determine, from the probability model, a probability score indicating a likelihood of the classification of the health condition being correct; and generate an output based on the health condition and the probability score.

Example 2. The medical system of Example 1, wherein the processing circuitry is configured to determine the respective one or more values for fluid retention by determining one or more values for impedance.

Example 3. The medical system of Example 1 or 2, wherein the plurality of fluid retention states comprises a wet state and a dry state.

Example 4. The medical system of any of Examples 1 through 3, wherein the plurality of temperature states comprises a cold state and a warm state.

Example 5. The medical system of any of Examples 1 through 4, wherein the output comprises the classification of the health condition.

Example 6. The medical system of any of Examples 1 through 5, wherein the output comprises a risk level associated with the health condition.

Example 7. The medical system of any of Examples 1 through 6, wherein the output comprises the probability score.

Example 8. The medical system of any of Examples 1 through 7, wherein the processing circuitry is configured to determine the respective one or more values for each of the plurality of physiological parameters by collecting a respective one or more measurements for each of the respective one or more values for each of the plurality of physiological parameters at a pre-determined time of day.

Example 9. The medical system of any of Examples 1 through 8, wherein the respective one or more values for fluid retention are determined from one or more subcutaneous tissue impedance measurements.

Example 10. The medical system of any of Examples 1 through 9, wherein the respective one or more values for fluid retention are determined from one or more tissue perfusion measurements.

Example 11. The medical system of any of Examples 1 through 10, wherein the respective one or more values for temperature are determined from one or more subcutaneous tissue temperature measurements, endocardial tissue temperature measurements, or intramuscular tissue temperature measurements.

Example 12. The medical system of any of Examples 1 through 11, wherein the processing circuitry is further configured to calibrate the respective one or more values for each of the plurality of physiological parameters.

Example 13. The medical system of Example 12, wherein the processing circuitry is configured to calibrate the respective one or more values for each of the plurality of physiological parameters by determining a respective median value for each of the respective one or more values for each of the plurality of physiological parameters.

Example 14. The medical system of Example 12 or 13, wherein the processing circuitry is configured to calibrate the respective one or more values for each of the plurality of physiological parameters by excluding, from the respective one or more values for each of the plurality of physiological parameters, a set of values from being used to identify the one or more diagnostic states, wherein the set of values fails to satisfy a noise threshold.

Example 15. The medical system of any of Examples 12 through 14, wherein the processing circuitry is configured to determine the respective one or more values for each of the plurality of physiological parameters based on a signal sensed by the implantable medical device, and wherein the processing circuitry is configured to calibrate the respective one or more values for the at least one physiological parameter by correcting the respective one or more values for of the at least one physiological parameters based on data transmitted from the external device.

Example 16. The medical system of Example 15, wherein the information comprises at least one of temperature data, acceleration data, patient-reported data, or location data.

Example 17. The medical system of Example 15 or 16, wherein the at least one of the physiological parameters comprises calibrated respective one or more values for temperature.

Example 18. The medical system of any of Examples 15 through 17, wherein the processing circuitry is configured to calibrate by performing principal component analysis or independent component analysis to extract, from the respective one or more values for each of the physiological parameters, a plurality of physiological parameters features.

Example 19. The medical system of any of Examples 1 through 18, wherein the probability score indicates a likelihood of a heart failure worsening event occurring within a predetermined timeframe.

Example 20. The medical system of any of Examples 1 through 19, further comprising the processing circuitry is further configured to generate an alert in response to the probability score satisfying a risk threshold.

Example 21. The medical system of any of Examples 1 through 20, wherein the processing circuitry is further configured to: compare the probability score to at least one risk threshold; and determine one of a plurality of discrete risk categorizations based on the comparison.

Example 22. The medical system of any of Examples 1 through 21, wherein the processing circuitry is configured to output the health condition and the probability score by transmitting the health condition and the probability score to another device.

Example 23. The medical system of any of Examples 1 through 22, wherein the processing circuitry is further configured to determine, for each of the plurality of physiological parameters, the respective one or more values at a plurality of frequencies.

Example 24. The medical system of any of Examples 1 through 23, wherein the processing circuitry is comprised in one or more of the implantable medical device, the external device, or the data server.

Example 25. The medical system of any of Examples 1 through 24, wherein the processing circuitry is external and separate from the implantable medical device, the external device, and the data server.

Example 26. A method comprising: determining, by processing circuitry of a medical device system, a respective one or more values for each of a plurality of physiological parameters, the plurality of physiological parameters comprising: fluid retention, wherein the respective one or more values for fluid retention correspond to a fluid retention state from a first set of states comprising a plurality of fluid retention states; and temperature, wherein the respective one or more values for temperature correspond to a temperature state from a second set comprising a plurality of temperature states; determining, by the processing circuitry, one or more diagnostic states based on the respective values; determining, by the processing circuitry, a classification of a health condition of a patient based on the application of the diagnostic states to the probability model; determining, by the processing circuitry and from the probability model, a probability score indicating a likelihood of the classification of the health condition being correct; and generating, by the processing circuitry, an output based on the health condition and the probability score.

Example 27. The method of Example 26, wherein determining the respective one or more values for fluid retention comprises determining one or more values for impedance.

Example 28. The method of Example 26 or 27 wherein the plurality of fluid retention states comprises a wet state and a dry state.

Example 29. The method of any of Examples 26 through 28, wherein the plurality of temperature states comprises a cold state and a warm state.

Example 30. The method of any of Examples 26 through 29, wherein the output comprises the classification of the health condition.

Example 31. The method of any of Examples 26 through 30, wherein the output comprises a risk level associated with the health condition.

Example 32. The method of any of Examples 26 through 31, wherein the output comprises the probability score.

Example 33. The method of any of Examples 26 through 32, wherein determining the respective one or more values for each of the plurality of physiological parameters comprises collecting a respective one or more measurements for each of the respective one or more values for each of the plurality of physiological parameters at a pre-determined time of day.

Example 34. The method of any of Examples 26 through 33, wherein the respective one or more values for fluid retention are determined from one or more subcutaneous tissue impedance measurements.

Example 35. The method of any of Examples 26 through 34, wherein the respective one or more values for fluid retention are determined from one or more tissue perfusion measurements.

Example 36. The method of any of Examples 26 through 35, wherein the respective one or more values for temperature are determined from one or more of subcutaneous tissue temperature measurements, endocardial tissue temperature measurements, or intramuscular tissue temperature measurements.

Example 37. The method of any of Examples 26 through 36, further comprising calibrating, by the processing circuitry, the respective one or more values for each of the plurality of physiological parameters.

Example 38. The method of Example 37, wherein calibrating the respective one or more values for each of the plurality of physiological parameters comprises determining a respective median value for each of the respective one or more values for each of the plurality of physiological parameters.

Example 39. The method of any of Examples 26 through 38, wherein calibrating the respective one or more values for each of the plurality of physiological parameters comprises excluding, from the respective one or more values for each of the plurality of physiological parameters, a set of values from being used to identify the one or more diagnostic states, wherein the set of values fails to satisfy a noise threshold.

Example 40. The method of any of Examples 26 through 39, wherein determining the respective one or more values for each of the plurality of physiological parameters comprises determining the one or more values for at least one of the physiological parameters based on a signal sensed by an implantable medical device, and wherein calibrating the respective one or more values for the at least one physiological parameter comprises correcting the respective one or more values for of the at least one physiological parameters based on data transmitted from an external device.

Example 41. The method of Example 40, wherein the information comprises at least one of temperature data, acceleration data, patient-reported data, or location data.

Example 42. The method of Example 40 or 41, Wherein the at least one of the physiological parameters comprises calibrated respective one or more values for temperature.

Example 43. The method of any of Examples 40 through 42, wherein calibrating comprises performing principal component analysis to extract, from the respective one or more values for each of the physiological parameters, a plurality of physiological parameters features.

Example 44. The method of any of Examples 26 through 43, wherein the probability score indicates a likelihood of a heart failure worsening event occurring within a predetermined timeframe.

Example 45. The method of any of Examples 26 through 44, further comprising generating, by the processing circuitry, an alert in response to the probability score satisfying a risk threshold.

Example 46. The method of any of Examples 26 through 45, further comprising: comparing, by the processing circuitry, the probability score to at least one risk threshold; and determining, by the processing circuitry, one of a plurality of discrete risk categorizations based on the comparison.

Example 47. The method of any of Examples 26 through 46, wherein outputting the health condition and the probability score comprises transmitting the health condition and the probability score to another device.

Example 48. The method of any of Examples 26 through 47, further comprising determining, by the processing circuitry and for each of the plurality of physiological parameters, the respective one or more values at a plurality of frequencies.

Example 49. The method of any of Examples 26 through 48, wherein the processing circuitry is comprised in one or more of the implantable medical device, the external device, or the data server.

Example 50. The method of any of Examples 26 through 49, wherein the processing circuitry is external and separate from the implantable medical device, the external device, and the data server.

Example 51. A computing device comprising processing circuitry configured to: determine a respective one or more values for each of a plurality of physiological parameters, the plurality of physiological parameters comprising: fluid retention, wherein the respective one or more values for fluid retention correspond to a fluid retention state from a first set of states comprising a plurality of fluid retention states; and temperature, wherein the respective one or more values for temperature correspond to a temperature state from a second set comprising a plurality of temperature states; determine, using a probability model, one or more diagnostic states based on the respective values; determine a classification of a health condition of a patient based on the application of the diagnostic states to the probability model; determine, from the probability model, a probability score indicating a likelihood of the classification of the health condition being correct; and generate an output based on the health condition and the probability score.

Example 51. The computing device of Example 51, wherein the processing circuitry is further configured to perform any of the methods of Examples 26 through 50.

1. A medical system comprising:
an implantable medical device;
an external device;
a data server; and
processing circuitry configured to:
determine a respective one or more values for each of a plurality of physiological parameters, the plurality of physiological parameters comprising:
fluid retention, wherein the respective one or more values for fluid retention correspond to a fluid retention state from a first set of states comprising a plurality of fluid retention states; and
temperature, wherein the respective one or more values for temperature correspond to a temperature state from a second set comprising a plurality of temperature states;
determine, using a probability model, one or more diagnostic states based on the respective values;
determine a classification of a health condition of a patient as one of normal or heart failure based on the application of the diagnostic states to the probability model;
determine, from the probability model, a probability score indicating a likelihood of the classification of the health condition being correct; and
generate an output based on the health condition and the probability score.

2. The medical system of claim 1, wherein the processing circuitry is configured to determine the respective one or more values for fluid retention by determining one or more values for impedance.

3. The medical system of claim 1, wherein the plurality of fluid retention states comprises a wet state and a dry state.

4. The medical system of claim 1, wherein the plurality of temperature states comprises a cold state and a warm state.

5. The medical system of claim 1, wherein the output comprises the classification of the health condition.

6. The medical system of claim 1, wherein the output comprises a risk level associated with the health condition.

7. The medical system of claim 1, wherein the output comprises the probability score.

8. The medical system of claim 1, wherein the processing circuitry is configured to determine the respective one or more values for each of the plurality of physiological parameters by collecting a respective one or more measurements for each of the respective one or more values for each of the plurality of physiological parameters at a pre-determined time of day.

9. The medical system of claim 1, wherein the respective one or more values for fluid retention are determined from one or more subcutaneous tissue impedance measurements.

10. The medical system of claim 1, wherein the respective one or more values for fluid retention are determined from one or more tissue perfusion measurements.

11. The medical system of claim 1, wherein the respective one or more values for temperature are determined from one or more subcutaneous tissue temperature measurements, endocardial tissue temperature measurements, or intramuscular tissue temperature measurements.

12. The medical system of claim 1, wherein the processing circuitry is further configured to calibrate the respective one or more values for each of the plurality of physiological parameters.

13. The medical system of claim 12, wherein the processing circuitry is configured to calibrate the respective one or more values for each of the plurality of physiological parameters by determining a respective median value for each of the respective one or more values for each of the plurality of physiological parameters.

14. A method comprising:
determining, by processing circuitry of a medical device system, a respective one or more values for each of a plurality of physiological parameters, the plurality of physiological parameters comprising:
fluid retention, wherein the respective one or more values for fluid retention correspond to a fluid retention state from a first set of states comprising a plurality of fluid retention states; and
temperature, wherein the respective one or more values for temperature correspond to a temperature state from a second set comprising a plurality of temperature states;
determining, by the processing circuitry, one or more diagnostic states based on the respective values;
determining, by the processing circuitry, a classification of a health condition of a patient as one of normal or heart failure based on the application of the diagnostic states to the probability model;
determining, by the processing circuitry and from the probability model, a probability score indicating a likelihood of the classification of the health condition being correct; and
generating, by the processing circuitry, an output based on the health condition and the probability score.

15. A computing device comprising processing circuitry configured to:
determine a respective one or more values for each of a plurality of physiological parameters, the plurality of physiological parameters comprising:
fluid retention, wherein the respective one or more values for fluid retention correspond to a fluid retention state from a first set of states comprising a plurality of fluid retention states; and
temperature, wherein the respective one or more values for temperature correspond to a temperature state from a second set comprising a plurality of temperature states;
determine, using a probability model, one or more diagnostic states based on the respective values;
determine a classification of a health condition of a patient as one of normal or heart failure based on the application of the diagnostic states to the probability model;
determine, from the probability model, a probability score indicating a likelihood of the classification of the health condition being correct; and
generate an output based on the health condition and the probability score.

16. The method of claim 14, wherein determining the respective one or more values for fluid retention comprises determining one or more values for impedance.

17. The method of claim 14, wherein the plurality of fluid retention states comprises a wet state and a dry state.

18. The method of claim 14, wherein determining the respective one or more values for each of the plurality of physiological parameters comprises collecting a respective one or more measurements for each of the respective one or more values for each of the plurality of physiological parameters at a pre-determined time of day.

19. The method of claim 14, further comprising calibrating, by the processing circuitry, the respective one or more values for each of the plurality of physiological parameters.

20. The method of claim 19, wherein calibrating the respective one or more values for each of the plurality of physiological parameters comprises determining a respective median value for each of the respective one or more values for each of the plurality of physiological parameters.
